(19) United States
(12) Patent Application Publication
(10) Pub. No.: US 2024/0218181 A1
(43) Pub. Date: Jul. 4, 2024

(54) SILK LEATHER AND RELATED MATERIALS, AND METHODS OF MAKING AND USING THE SAME

(71) Applicant: Trustees of Tufts College, Medford, MA (US)

(72) Inventors: Marco Lo Presti, Arlington, MA (US); Barbara Pollini, Graffignana (Lodi) (IT); Fiorenzo G. Omenetto, Lexington, MA (US)

(21) Appl. No.: 18/403,477

(22) Filed: Jan. 3, 2024

Related U.S. Application Data
(63) Continuation of application No. PCT/US23/83663, filed on Dec. 12, 2023.
(60) Provisional application No. 63/387,066, filed on Dec. 12, 2022.
(60) Provisional application No. 63/495,729, filed on Apr. 12, 2023.

(51) Int. Cl. C08L 89/00; A61K 47/42; C08J 9/00; C08J 9/30; C08K 5/053; C08L 5/00; D06N 3/00

(52) U.S. Cl. CPC C08L 89/00; A61K 47/42; C08J 9/0023; C08J 9/0061; C08J 9/30; C08K 5/053; C08L 5/00; D06N 3/0045; D06N 3/0061; C08J 2305/00; C08J 2389/00; C08L 2203/14; C08L 2205/06; D06N 2205/04

(57) ABSTRACT

A host of new silk material formats are disclosed, all prepared from an initial liquid composition including both silk fibroin and xanthan gum. In some cases, particularly preferred material properties are achieved by the addition of glycerol. These various formats include liquids, whipped silk creams, silk meringues, and compressed/heat-compressed meringues. Among the useful end-uses for these materials is a silk leather, which mimics the feel of conventional leather. Other uses include thermal insulation, absorption/gas sensing, biological scaffolding, and the like. A material that has material properties unexpectedly similar to mycelium is also disclosed.

### CROSS-REFERENCE TO RELATED APPLICATIONS

This application is a bypass continuation of International Application Serial Number PCT/US2023/083663, filed Dec. 12, 2023. International Application Serial Number PCT/US2023/083663 is related to, claims priority to, and incorporates herein by reference for all purposes both of: U.S. Provisional Pat. App. No. 63/387,066, filed Dec. 12, 2022; and U.S. Provisional Pat. App. No. 63/495,729, filed Apr. 12, 2023.

### BACKGROUND

Sustainability in materials is an area of increasing interest. One material that is sustainable in a certain sense is leather, in that it can be reproduced and grown from animals. However, this material is not sustainable when it comes to humane treatment of animals. Alternatives to leather have been pursued for decades, but those alternatives have primarily been non-sustainable alternatives.

A need exists for alternatives to conventional leather as a material for use across the spectrum of applications where conventional leather is used.

### SUMMARY

In one aspect, the present disclosure provides a liquid composition comprising a mixture of silk fibroin and xanthan gum.

In another aspect, the present disclosure provides a whipped silk cream comprising silk fibroin and xanthan gum.

In a further aspect, the present disclosure provides a silk meringue comprising silk fibroin and xanthan gum.

In yet another aspect, the present disclosure provides a compressed or hot-pressed silk meringue comprising silk fibroin and xanthan gum.

In another aspect, the present disclosure provides methods of making a composition. To make a whipped silk cream, the method involves whipping the liquid composition comprising silk fibroin and xanthan gum (and optionally glycerol) for a predetermined whipping time to form a whipped silk cream. To make a silk meringue, the method comprises baking the whipped silk cream. To make a compressed silk meringue, the method involves compressing the silk meringue. To make a hot-pressed silk meringue, the method involves hot-pressing the silk meringue.

In yet another aspect, the present disclosure provides a silk leather. The silk leather is a layer structure including a first fabric layer and a second material layer disposed adjacent to the first fabric layer. The second material layer is or includes the compressed or hot-pressed silk meringue disclosed herein.

In an aspect, a method of making a whipped silk cream having a desired whipped silk cream morphology, a silk meringue having a desired silk meringue morphology, a compressed silk meringue having a desired compressed silk meringue morphology, or a hot-pressed silk meringue having a desired hot-pressed silk meringue morphology may include selecting a silk concentration, a silk molecular weight distribution, a polysaccharide species, a polysaccharide concentration, a plasticizer species, a plasticizer concentration, a whipping speed, optionally a silk meringue baking temperature, optionally a compressed silk meringue compressing force, optionally a hot-pressed silk meringue hot-pressing force and temperature; and making the whipped silk cream, the silk meringue, the compressed silk meringue, or the hot-pressed silk meringue using the silk concentration, the silk molecular weight distribution, the polysaccharide species, the polysaccharide concentration, the plasticizer species, the plasticizer concentration, the whipping speed, optionally the silk meringue baking temperature, optionally the compressed silk meringue compressing force, and optionally the hot-pressed silk meringue hot-pressing force and temperature.

In some aspects, the techniques described herein relate to a liquid composition including a mixture of silk fibroin, xanthan gum, and a plasticizer.

In some aspects, the techniques described herein relate to a whipped silk cream including silk fibroin, xanthan gum, and a plasticizer.

In some aspects, the techniques described herein relate to a silk meringue including silk fibroin, xanthan gum, and a plasticizer.

In some aspects, the techniques described herein relate to a compressed silk meringue including silk fibroin, xanthan gum, and a plasticizer.

In some aspects, the techniques described herein relate to a hot-pressed silk meringue including silk fibroin, xanthan gum, and a plasticizer.

In some aspects, the techniques described herein relate to a method of making a composition, the method including: whipping a liquid including silk fibroin, xanthan gum, and a plasticizer for a predetermined whipping time to form a whipped silk cream.

In some aspects, the techniques described herein relate to a liquid composition including a mixture of silk fibroin, xanthan gum, and a functionalizing agent.

In some aspects, the techniques described herein relate to a whipped silk cream including silk fibroin, xanthan gum, and a functionalizing agent.

In some aspects, the techniques described herein relate to a silk meringue including silk fibroin, xanthan gum, and a functionalizing agent.

In some aspects, the techniques described herein relate to a compressed silk meringue including silk fibroin, xanthan gum, and a functionalizing agent.

In some aspects, the techniques described herein relate to a hot-pressed silk meringue including silk fibroin, xanthan gum, and a functionalizing agent.

In some aspects, the techniques described herein relate to a method of making a composition, the method including whipping a liquid including silk fibroin, xanthan gum, and a functionalizing agent for a predetermined whipping time to form a whipped silk cream.

In some aspects, the techniques described herein relate to a liquid composition including a mixture of silk fibroin, xanthan gum, a plasticizer, and a functionalizing agent.

In some aspects, the techniques described herein relate to a whipped silk cream including silk fibroin, xanthan gum, a plasticizer, and a functionalizing agent.

In some aspects, the techniques described herein relate to a silk meringue including silk fibroin, xanthan gum, a plasticizer, and a functionalizing agent.

In some aspects, the techniques described herein relate to a compressed silk meringue including silk fibroin, xanthan gum, a plasticizer, and a functionalizing agent.

In some aspects, the techniques described herein relate to a hot-pressed silk meringue including silk fibroin, xanthan gum, a plasticizer, and a functionalizing agent.

In some aspects, the techniques described herein relate to a method of making a composition, the method including: whipping a liquid including silk fibroin, xanthan gum, a plasticizer, and a functionalizing agent for a predetermined whipping time to form a whipped silk cream.

In some aspects, the techniques described herein relate to a conductive silk leather.

In some aspects, the techniques described herein relate to a magnetic silk leather.

In some aspects, the techniques described herein relate to a scented silk leather.

In some aspects, the techniques described herein relate to a thermally-insulating silk leather having thermochromic reporting property throughout a bulk interior volume.

In some aspects, the techniques described herein relate to a pH responsive leather including a pH responsive chemical distributed throughout the compressed silk meringue or the hot-pressed silk meringue of the pH responsive leather.

In some aspects, the techniques described herein relate to a humidity sensing leather including a pH responsive chemical and a pH altering agent distributed throughout the compressed silk meringue or the hot-pressed silk meringue of the pH responsive leather, wherein measurable amounts of humidity solubilize at least a portion of the pH altering agent, thereby lowering the pH, thereby providing a measurable report of humidity.

In some aspects, the techniques described herein relate to a patterned silk leather.

In some aspects, the techniques described herein relate to an electronic leather having an electronic component embedded therein.

In some aspects, the techniques described herein relate to the electronic silk leather of the immediately preceding claim, wherein the power supply is a rechargeable battery, a wired disposable batter holder, or a combination thereof.

In some aspects, the techniques described herein relate to a semiconductor device-embedded silk leather having a semiconductor device embedded therein.

In some aspects, the techniques described herein relate to a haptic silk leather having a haptic switch embedded therein.

In some aspects, the techniques described herein relate to a tanned silk leather, the tanned silk leather having a bulk volume of a compressed silk meringue or hot-compressed silk meringue and a surface layer of the compressed silk meringue or hot-pressed silk meringue, wherein the surface layer is formed from the same chemical composition as the bulk volume but includes at least one differing structural, mechanical, or chemical feature relative to the bulk volume.

In some aspects, the techniques described herein relate to a whipped silk cream or silk meringue having fibers distributed throughout.

In some aspects, the techniques described herein relate to a whipped silk cream or silk meringue having electronics distributed throughout.

In some aspects, the techniques described herein relate to an ultra lightweight silk down alternative including silk meringue.

In some aspects, the techniques described herein relate to an impact-distributing foam including a silk meringue.

In some aspects, the techniques described herein relate to a protected item including: an item to be protected; and a protective shell including a silk meringue, a cured silk meringue, a compressed silk meringue, or a hot-compressed silk meringue, wherein the protective shell is formed by surrounding and contacting the item to be protected with a precursor to the protective shell and curing the precursor to form the protective shell, wherein surrounding includes fully encapsulating or encapsulating against a surface.

In some aspects, the techniques described herein relate to an open-cell silk foam made by whipping a liquid silk composition including silk fibroin and xanthan gum past a first overrun value and short of a second overrun value to form a silk whipped cream with an open structure; and subsequently baking the silk whipped cream to make the open-cell silk foam.

In some aspects, the techniques described herein relate to a closed-cell silk foam made by whipping a liquid silk composition including silk fibroin and xanthan gum either short of a first overrun value or past a second overrun value to form a silk whipped cream with a closed structure; and subsequently baking the silk whipped cream to make the closed-cell silk foam.

In some aspects, the techniques described herein relate to a liquid composition including a mixture of a protein, a polysaccharide, and a plasticizer.

In some aspects, the techniques described herein relate to a whipped silk cream including silk fibroin, a polysaccharide, and a plasticizer.

In some aspects, the techniques described herein relate to a silk meringue including silk fibroin, a polysaccharide, and a plasticizer.

In some aspects, the techniques described herein relate to a compressed silk meringue including silk fibroin, a polysaccharide, and a plasticizer.

In some aspects, the techniques described herein relate to a hot-pressed silk meringue including silk fibroin, a polysaccharide, and a plasticizer.

In some aspects, the techniques described herein relate to a method of making a composition, the method including whipping a liquid composition including a protein, a polysaccharide, and a plasticizer for a predetermined whipping time to form a whipped silk cream.

In some aspects, the techniques described herein relate to a liquid composition including a mixture of silk fibroin, an alginate, and a plasticizer.

In some aspects, the techniques described herein relate to a whipped silk cream including silk fibroin, an alginate, and a plasticizer.

In some aspects, the techniques described herein relate to a silk meringue including silk fibroin, an alginate, and a plasticizer.

In some aspects, the techniques described herein relate to a compressed silk meringue including silk fibroin, an alginate, and a plasticizer.

In some aspects, the techniques described herein relate to a hot-pressed silk meringue including silk fibroin, an alginate, and a plasticizer.

In some aspects, the techniques described herein relate to a method of making a composition, the method including: whipping a liquid including silk fibroin, an alginate, and a plasticizer for a predetermined whipping time to form a whipped silk cream. 363.

### BRIEF DESCRIPTION OF THE FIGURES

The disclosure and the following detailed description of certain aspects thereof may be understood by reference to the following figures:

FIGS. 1A and 1B depict silk leather of the disclosure.

FIG. 1C depicts a CO₂ sensing reaction.

FIG. 2 depicts a mechanical comparison between Mycelium and Mysilkium.

FIG. 3 depicts mechanical properties of composite mysilkium and relative textile.

FIG. 4 depicts cream density.

FIG. 5 depicts cream water content.

FIG. 6 depicts meringue density.

FIG. 7 shows the solid compositions of the meringues obtained by varying the mass of SF and assuming a total removal of water from the cooking process.

FIG. 8 provides data regarding various compositions and the ratio of SF. Gly, and XG in each composition.

FIG. 9a depicts the cream density for the compositions identified in FIG. 8.

FIG. 9b depicts the cream water content for the compositions identified in FIG. 8.

FIG. 10a depicts the syneresis (%) for the compositions identified in FIG. 8 and FIG. 10b depicts the relationship of density, water content, and syneresis to solid content.

FIG. 11a depicts density variation of the cream as a function of whipping time.

FIG. 11b depicts the mechanical properties of the cream and their variation with whipping time through resistance toward compression (firmness).

FIG. 12 depicts overrun of the creams measured at different whipping times and using different plasticizers.

FIG. 13 depicts that the density of the cream reduces and can be controlled through whipping time.

FIG. 14 depicts the transition from random coils to β-sheet structure upon whipping.

FIG. 15 depicts the relationship of bubble size and overrun.

FIG. 16a depicts a photograph of SF:XG:Gly dried foam; FIG. 16b depicts densities; FIG. 16C depicts compressive strength; and FIG. 16D depicts the yield point using different ratios for SF:XG:Gly dried foams.

FIG. 17A depicts a comparison of the mechanical performance of xanthan and alginate-based silk foams FIG. 17B depicts a comparison of the firmness of xanthan and alginate-containing silk creams; FIG. 17C depicts an experimental setup; and FIG. 17D depicts a temperature vs time plot of alginate foam vs. polystyrene.

FIG. 18 depicts photographs (A) and densities (B) of SF:XG:Gly 20:20:60 foams at different minutes of whipping.

FIG. 19A depicts photographs with the detail of the surface morphology of the foam and FIG. 19B) depicts its internal structure as a function of the whipping time obtained through fluorescent staining with rhodamine 6G.

FIG. 20 depicts foams fluorescent staining (ThT) to analyze the internal foam structure.

FIG. 21A depicts spectral features for ThT free in solution when excited at 365 nm and FIG. 21B depicts spectral features for ThT bound to a β-sheet structure when excited at 365 nm.

FIG. 22 depicts the emission of foams excited at 365 nm displaying a color shift during the initial whipping phases.

FIGS. 23A, 23B, and 23C depict foam materials as a substrate for algal growth.

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

### Definitions

As used herein, “low molecular weight” silk fibroin refers to silk fibroin that has been subjected to boiling during degumming or another processing step for a length of time of at least 30 minutes, thereby reducing the average molecular weight of the protein fragments. Examples of low molecular weight silk fibroin can be found at WO 2014/145002, which is incorporated herein in its entirety by reference.

As used herein, mysilkium refers to a material that is composed of at least 20% silk fibroin and which has one or more of the following material properties falling within 50%, within 25%, within 20%, within 15%, or within 10% of a native mycelium.

As used herein, “silk fibroin” refers to silk fibroin protein whether produced by silkworm, spider, or other insect, or otherwise generated (Lucas et al., Adv. Protein Chem., 13: 107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. Sec, e.g., WO 97/08315 and U.S. Pat. No. 5,245,012, each of which is incorporated herein by reference in their entireties.

### Liquid Compositions

The present disclosure provides a liquid composition. Compositions disclosed herein may include a protein, at least one polysaccharide, and a plasticizer. In embodiments, the protein may be silk fibroin. In embodiments, the polysaccharide may be xanthan gum, an alginate, or another high molecular weight sugar, cellulose derivative, combinations thereof, or the like. Liquid compositions containing xanthan gum may be superior at obtaining silk leathers. It should be understood, however, that examples and embodiments throughout this Specification that reference xanthan gum as a component may comprise a different polysaccharide in place of, or in addition to, xanthan gum, such as an alginate. In some embodiments, selection of the particular polysaccharide or combination of polysaccharides used in the liquid composition may depend on the ultimate material properties desired. For example, in some aspects, alginate may be preferred if a free-standing leather material is desired. It is expressly contemplated that certain applications may require a combination of different polysaccharides within the same composition, such as a specific application requiring a whipped silk cream including silk fibroin, glycerol, xanthan gum, and alginate as the principle components.

In some aspects, the weight ratio of silk fibroin, polysaccharide, and plasticizer may have an impact on one or more of a mechanical property, a density, or a water content of a resulting material made from the composition. Without wishing to be bound by any particular theory, variations of the weight of the plasticizer may preferentially impact mechanical properties, variations of the weight of the polysaccharide or combination of polysaccharides may preferentially impact density, and variations of the weight of the silk fibroin may preferentially impact water content.

In some embodiments, the liquid composition includes a mixture of silk fibroin and xanthan gum. In some aspects, the liquid composition includes a mixture of silk fibroin, xanthan gum, and a plasticizer. In some aspects, the liquid composition includes a mixture of silk fibroin, xanthan gum, and glycerol. In some aspects, the liquid composition includes a mixture of silk fibroin, xanthan gum, and a functionalizing agent. In some aspects, the liquid composition includes a mixture of silk fibroin, xanthan gum, a plasticizer, and a functionalizing agent. In some aspects, the liquid composition includes a mixture of silk fibroin, xanthan gum, glycerol, and a functionalizing agent. In some aspects, the liquid composition includes a mixture of silk fibroin and a high molecular weight sugar (e.g., such as xanthan gum), and may optionally further include at least one of a plasticizer (e.g., such as glycerol) or a functionalizing agent.

The liquid composition can include other components that a skilled artisan will recognize are valuable in certain contexts. In certain cases, the liquid composition can further include glycerol. In certain cases, the liquid composition can further include a sensing agent. In certain case, the liquid composition can further include a therapeutically active agent. In certain cases, the liquid composition can include an aroma-providing compound.

The mixture of silk fibroin and xanthan gum includes a weight ratio of silk fibroin to xanthan gum of between 1:4 and 20:1 or between 1:2 and 10:1. For example, the mixture of silk fibroin and xanthan gum can include a weight ratio of silk fibroin to xanthan gum of at least 1:4, at least 1:3, or at least 1:2. For example, mixture of silk fibroin and xanthan gum can include a weight ratio of silk fibroin to xanthan gum of at most 20:1, at most 19:1, at most 18:1, at most 16:1, at most 15:1, at most 14:1, at most 12:1, at most 11:1, or at most 10:1.

The silk fibroin can be present in the liquid composition in an amount by weight of between 1% and 10% or between 3% and 7%. Without wishing to be bound by any particular theory, it is believed that the concentration of silk fibroin can impact the structural integrity of a resulting product. If the concentration is too low, the resulting product may not coalesce into a single physical structure, thereby failing to make an article at all. If the concentration is too high, the resulting product may form in a fashion with visible defects, cracks, and other imperfections. For example, the silk fibroin can be present in the liquid composition in an amount by weight of at least 1%, at least 2%, at least 3%, at least 4%, at least 5%, or at least 6%. For example, the silk fibroin can be present in the liquid composition in an amount by weight of at most 10%, at most 9%, at most 8%, at most 7%, at most 6%, or at most 5%.

The xanthan gum can be present in the liquid composition in an amount by weight of between 0.1% and 10.0%. For example, the xanthan gum can be present in the liquid composition in an amount by weight of at least 0.1%, at least 0.3%, at least 0.5%, at least 1.0%, at least 1.5%, at least 2.0%, at least 2.5%, at least 3.0%, at least 3.5%, at least 4.0%, at least 4.5%, at least 5.0%, at least 5.5%, at least 6.0%, at least 6.5%, at least 7.0%, at least 7.5%, at least 8.0%, at least 8.5%, at least 9.0%, or at least 9.5%. For example, the xanthan gum can be present in the liquid composition in an amount by weight of at most 10.0%, at most 9.5%, at most 9.0%, at most 8.5%, at most 8.0%, at most 7.5%, at most 7.0%, at most 6.5%, at most 6.0%, at most 5.5%, at most 5.0%, at most 4.5%, at most 4.0%, at most 3.5%, at most 3.0%, at most 2.5%, at most 2.0%, at most 1.5%, or at most 1.0%.

The plasticizer can be present in the liquid composition in an amount by weight of between 0.5% and 20.0%. For example, the plasticizer can be present in the liquid composition in an amount by weight of at least 0.5%, at least 1.0%, at least 1.5%, at least 2.0%, at least 2.5%, at least 3.0%, at least 3.5%, at least 4.0%, at least 4.5%, at least 5.0%, at least 5.5%, at least 6.0%, at least 6.5%, at least 7.0%, at least 7.5%, at least 8.0%, at least 8.5%, at least 9.0%, at least 9.5%, at least 10.0%, at least 12.0%, or at least 15.0%. For example, the plasticizer can be present in the liquid composition in an amount by weight of at most 20.0%, at most 18.5%, at most 17.5%, at most 16.0%, at most 15.0%, at most 14.0%, at most 13.0%, at most 12.5%, at most 11.0%, at most 10.0%, at most 8.0%, or at most 5.0%.

The plasticizer can be present in the liquid composition in an amount by weight of between 20.0% and 75.0%. For example, the plasticizer can be present in the liquid composition in an amount by weight of at least 20.0%, at least 25.0%, at least 30.0%, at least 35.0%, at least 40.0%, at least 45.0%, at least 50.0%, at least 55.0%, at least 60.0%, at least 65.0%, or at least 70.0%. For example, the plasticizer can be present in the liquid composition in an amount by weight of at most 75.0%, at most 70.0%, at most 65.0%, at most 60.0%, at most 55.0%, at most 50.0%, at most 45.0%, at most 40.0%, at most 35.0%, at most 30.0%, at most 25.0%, or at most 20.0%.

Plasticizers can include at least one of polyols, esters, phthalates, terephthalates, trimellitates, adipates, sebacates, organophosphates, ethanolamines, or glycerols. The plasticizer can be selected from the group consisting of glycerol, 1,2 pentanediol, 1,5 pentanediol, 1,2,6 hexanetriol, and mixtures thereof. In some cases, the plasticizer is glycerol. In some cases, the plasticizer is 1,2 pentanediol. In some cases, the plasticizer is 1,5 pentanediol. In some cases, the plasticizer is 1,2,6 hexanetriol.

Without wishing to be bound by any particular theory, it is believed that the number or and/or separation between —OH substituents can strongly impact the material properties of the resulting composition. In some cases, the plasticizer comprises at least one —OH substituent. In some cases, the plasticizer comprises at least two —OH substituents, at least 3 —OH substituents, or more —OH substituents. In some cases, the at least two —OH substituents, the at least 3 —OH substituents, or the more —OH substituents are separated from one another on the plasticizer by at least 2 carbon atoms, at least 3 carbon atoms, or at least 4 carbon atoms.

In some cases, the plasticizer can be partly or wholly evaporated off during processing. In some cases, the plasticizer remains within the composition during processing.

The sensing agent can be present in an amount that is selected by the nature of the sensing agent and the nature of the desired sensing performance. Similarly, a therapeutically active agent can be present in an amount that is selected by the nature of the therapeutically active agent and the nature of the desired therapeutic outcome. Similarly, a colorant can be present in an amount that is selected by the nature of the coloring ability of the colorant and the nature of the desired coloring. Similarly, an aroma-providing compound can be present in an amount that is selected by the nature of the aroma-providing ability of the compound and the nature of the desired aroma performance. A skilled artisan will recognize that there will be differing lower and upper boundaries for the amount of sensing agent, therapeutically active agent, colorant, and/or aroma-providing compound that will be present based on the nature of the agent. The aforementioned additives, agents, colorants, etc. can optionally be food-safe versions, which are identified as generally recognized as safe according to the US Food and Drug Administration. The additive can be inorganic, with uses such as fluorescent materials, lasing materials/media, absorbing/light responsive materials, temperature-sensitive materials, electrochemical materials, or combinations thereof.

In some aspects of the liquid composition or any of the downstream products or articles of manufacture disclosed herein, between 3% and 72% of the total weight of the silk fibroin, the xanthan gum, and the glycerol is the silk fibroin. In some aspects of the liquid composition or any of the downstream products or articles of manufacture disclosed herein, between 3% and 32% of the total weight of the silk fibroin, the xanthan gum, and the glycerol is the xanthan gum. In some aspects of the liquid composition or any of the downstream products or articles of manufacture disclosed herein, between 25% and 94% of the total weight of the silk fibroin, the xanthan gum, and the glycerol is the glycerol. In some aspects of the liquid composition or any of the downstream products or articles of manufacture disclosed herein, between 3% and 32% of the total weight of the silk fibroin, the xanthan gum, and the glycerol is the xanthan gum, and between 25% and 94% of the total weight of the silk fibroin, the xanthan gum, and the glycerol is the glycerol.

In some aspects of the liquid composition or any of the downstream products or articles of manufacture disclosed herein, between 15% and 25% of the total weight of the silk fibroin, the xanthan gum, and the glycerol is the silk fibroin, between 50% and 70% of the total weight of the silk fibroin, the xanthan gum, and the glycerol is the glycerol, and between 15% and 25% of the total weight of the silk fibroin, the xanthan gum, and the glycerol is the xanthan gum.

In some aspects of the liquid composition or any of the downstream products or articles of manufacture disclosed herein, between 15% and 25% of the total weight of the silk fibroin and the glycerol is the silk fibroin, and between 75% and 85% of the total weight of the silk fibroin and the glycerol is the glycerol.

In some aspects of the liquid composition or any of the downstream products or articles of manufacture disclosed herein, between 30% and 35% of the total weight of the silk fibroin, the xanthan gum, and the glycerol is the silk fibroin, between 30% and 35% of the total weight of the silk fibroin, the xanthan gum, and the glycerol is the glycerol, and between 30% and 35% of the total weight of the silk fibroin, the xanthan gum, and the glycerol is the xanthan gum.

In some aspects of the liquid composition or any of the downstream products or articles of manufacture disclosed herein, between 35% and 45% of the total weight of the silk fibroin, the xanthan gum, and the glycerol is the silk fibroin, between 15% and 25% of the total weight of the silk fibroin, the xanthan gum, and the glycerol is the glycerol, and between 35% and 45% of the total weight of the silk fibroin, the xanthan gum, and the glycerol is the xanthan gum.

In some aspects of the liquid composition or any of the downstream products or articles of manufacture disclosed herein, between 15% and 25% of the total weight of the silk fibroin, the xanthan gum, and the glycerol is the silk fibroin, between 35% and 45% of the total weight of the silk fibroin, the xanthan gum, and the glycerol is the glycerol, and between 35% and 45% of the total weight of the silk fibroin, the xanthan gum, and the glycerol is the xanthan gum.

In some aspects of the liquid composition or any of the downstream products or articles of manufacture disclosed herein, between 35% and 45% of the total weight of the silk fibroin, the xanthan gum, and the glycerol is the silk fibroin, between 35% and 45% of the total weight of the silk fibroin, the xanthan gum, and the glycerol is the glycerol, and between 15% and 25% of the total weight of the silk fibroin, the xanthan gum, and the glycerol is the xanthan gum.

In some aspects of the liquid composition or any of the downstream products or articles of manufacture disclosed herein, between 45% and 55% of the total weight of the silk fibroin and the xanthan gum is the silk fibroin, and between 45% and 55% of the total weight of the silk fibroin and the xanthan gum is the xanthan gum.

In some aspects of the liquid composition or any of the downstream products or articles of manufacture disclosed herein, between 75% and 85% of the total weight of the silk fibroin and the xanthan gum is the silk fibroin, and between 15% and 25% of the total weight of the silk fibroin and the xanthan gum is the xanthan gum.

In some aspects of the liquid composition or any of the downstream products or articles of manufacture disclosed herein, between 15% and 25% of the total weight of the silk fibroin, the xanthan gum, and the glycerol is the silk fibroin, between 15% and 25% of the total weight of the silk fibroin, the xanthan gum, and the glycerol is the glycerol, and between 55% and 65% of the total weight of the silk fibroin, the xanthan gum, and the glycerol is the xanthan gum.

The liquid composition has a high water content. In some cases, the liquid composition has a water content of between 89% and 99%.

### Whipped Silk Creams

Much like dairy milk can be whipped into a cream because of the emulsion-forming capacity of its high fat content, unexpectedly, silk fibroin solutions can be whipped up to a foam in the presence of high molecular weight sugars, such as xanthan gum or alginates. Foams obtained this way may be brittle and may display higher wettability towards organic solvents. Adding plasticizers to the composition may remove the brittleness and makes the foams more hydrophilic and encourages sponge-like behavior in water environments.

The present disclosure provides a whipped silk cream comprising silk fibroin and xanthan gum. In some aspects, the whipped silk cream comprises silk fibroin, xanthan gum, and a plasticizer. In some aspects, the whipped silk cream comprises silk fibroin, xanthan gum, and glycerol. In some aspects, the whipped silk cream comprises silk fibroin, xanthan gum, a plasticizer, and a functionalizing agent. In some aspects, the whipped silk cream comprises silk fibroin, xanthan gum, glycerol, and a functionalizing agent. In some aspects, the whipped silk cream comprises silk fibroin, xanthan gum, and a functionalizing agent. In some aspects, the whipped silk cream comprises a mixture of silk fibroin and a high molecular weight sugar (e.g., such as xanthan gum), and may optionally further include at least one of a plasticizer (e.g., such as glycerol) or a functionalizing agent.

Compositionally, the whipped silk cream can contain the same components in the same amounts as the liquid composition, with the liquid composition being transformed by the whipping process into a whipped silk cream.

The whipped silk cream can have an irregular porosity.

In some cases, the whipped silk cream can have an overrun that is comparable to the overrun of dairy whipped cream. Certain properties of the whipped silk cream may be associated with particular overrun values. In some aspects, the properties of the whipped silk cream at a particular overrun value may be variable based on if the particular overrun value is achieved before or after achieving a maximum overrun value. Maximum overrun values may depend on at least one of: the components included in the liquid composition, the weight ratio of one or more components in the liquid composition, the weight % of one or more components in the liquid composition, the amount of whipping time, or the temperature during whipping. In one example, liquid compositions including plasticizers may exhibit greater overrun relative to compositions lacking or having reduced amounts of plasticizers.

The whipped silk cream may exhibit an overrun of between 10% and 550%, between 20% and 550%, between 50% and 300%, between 10% and 100%, between 10% and 150%, between 10% and 300%, or between 100% and 300%. The whipped silk cream may exhibit an overrun of at least 10%, at least 50%, at least 100%, at least 200%, at least 300%, at least 400%, or at least 500%.

Given that the whipped silk cream is a downstream product from the liquid composition, the whipped silk cream can include any component or feature of the liquid composition, unless the context clearly dictates otherwise (e.g., if the feature relates specifically to being a liquid and not whipped).

The whipped silk cream has a water content that can be tailored for specific uses. In some cases, the water content of the whipped silk cream is between 50% and 95%, including but not limited to, between 75% and 93%, between 87% and 92%, or between 88.5% and 91%, including non-recited combinations of the upper and lower limits of those ranges (e.g., between 88.5% and 95%, etc.).

### Silk Meringue

The present disclosure provides a silk meringue comprising silk fibroin and xanthan gum. In some aspects, the silk meringue comprises silk fibroin, xanthan gum, and a plasticizer. In some aspects, the silk meringue comprises silk fibroin, xanthan gum, and glycerol. In some aspects, the silk meringue comprises silk fibroin, xanthan gum, a plasticizer, and a functionalizing agent. In some aspects, the silk meringue comprises silk fibroin, xanthan gum, glycerol, and a functionalizing agent. In some aspects, the silk meringue comprises silk fibroin, xanthan gum, and a functionalizing agent. In some aspects, the silk meringue comprises a mixture of silk fibroin and a high molecular weight sugar (e.g., such as xanthan gum), and may optionally further include at least one of a plasticizer (e.g., such as glycerol) or a functionalizing agent.

The silk meringue is a baked whipped silk cream. The silk meringue may be alternatively described as a foam herein. Compositionally, the silk meringue can contain the same components in the same amounts as the liquid composition and the whipped silk cream, with significantly less water/moisture content. Without wishing to be bound by any particular theory, it is believed that the whipping process and baking process can both give distinct characteristics to the silk meringue disclosed herein, with unique pore structure and size being generated by varying compositional and/or processing parameters.

Given that the silk meringue is a downstream product from the whipped silk cream, the silk meringue can include any component or feature of the whipped silk cream, unless the context clearly dictates otherwise (e.g., the water content is much lower in silk meringues). Similarly, as both the silk meringue and the whipped silk cream derive from the liquid composition, the silk meringue can include any component or feature of the liquid composition, unless the context clearly dictates otherwise.

The silk meringue has a water content that can be tailored for specific uses. The silk meringue can have a water content of between 5% and 70%.

### Compressed Silk Meringue

The present disclosure provides a compressed silk meringue comprising silk fibroin and xanthan gum and, in many cases, glycerol. In some aspects, the compressed silk meringue comprises silk fibroin, xanthan gum, and a plasticizer. In some aspects, the compressed silk meringue comprises silk fibroin, xanthan gum, and glycerol. In some aspects, the compressed silk meringue comprises silk fibroin, xanthan gum, a plasticizer, and a functionalizing agent. In some aspects, the compressed silk meringue comprises silk fibroin, xanthan gum, glycerol, and a functionalizing agent. In some aspects, the compressed silk meringue comprises silk fibroin, xanthan gum, and a functionalizing agent. In some aspects, the compressed silk meringue comprises a mixture of silk fibroin and a high molecular weight sugar (e.g., such as xanthan gum), and may optionally further include at least one of a plasticizer (e.g., such as glycerol) or a functionalizing agent.

Compositionally, the compressed silk meringue is generally the same as the silk meringue. Structurally, the compressed silk meringue has a reduced and/or compressed and/or damaged pore structure when compared with the silk meringue.

Many of the most preferred compressed silk meringues include glycerol, as its inclusion provides an impressive malleability, thereby allowing compression with the retention of the general material and pore structure of the silk meringue. Specifically, in some cases where glycerol is present, the compressed silk meringue is a mysilkium material. Specifically, in some cases where glycerol is present, the compressed silk meringue can be or can form a part of (e.g., one or two layers adhered to a fabric substrate) a silk leather. In an embodiment, meringues disclosed herein can be used as an alternative to polyurethane foams employed for artificial leathers.

Given that the compressed silk meringue is a downstream product from the silk meringue, the compressed silk meringue can include any component or feature of the silk meringue, unless the context clearly dictates otherwise (e.g., the porosity is reduced in the compressed silk meringue). Similarly, as all of the compressed silk meringue, the silk meringue, and the whipped silk cream derive from upstream entities, the compressed silk meringue can include any component or feature of the silk meringue, the whipped silk cream, or the liquid composition, unless the context clearly dictates otherwise.

The compressed silk meringue has a water content that can be tailored for specific uses. The compressed silk meringue has a water content of between 2% and 50%.

### Hot-Pressed Silk Meringue

The present disclosure provides a hot-pressed silk meringue comprising silk fibroin and xanthan gum and, in many cases, glycerol. In some aspects, the hot-pressed silk meringue comprises silk fibroin, xanthan gum, and glycerol. In some aspects, the hot-pressed silk meringue comprises silk fibroin, xanthan gum, and a plasticizer. In some aspects, the hot-pressed silk meringue comprises silk fibroin, xanthan gum, and glycerol. In some aspects, the hot-pressed silk meringue comprises silk fibroin, xanthan gum, a plasticizer, and a functionalizing agent. In some aspects, the hot-pressed silk meringue comprises silk fibroin, xanthan gum, glycerol, and a functionalizing agent. In some aspects, the hot-pressed silk meringue comprises silk fibroin, xanthan gum, and a functionalizing agent. In some aspects, the hot-pressed silk meringue comprises a mixture of silk fibroin and a high molecular weight sugar (e.g., such as xanthan gum), and may optionally further include at least one of a plasticizer (e.g., such as glycerol) or a functionalizing agent.

Compositionally, the hot-pressed silk meringue is generally the same as the silk meringue. Structurally, the hot-pressed silk meringue has a reduced and/or compressed and/or damaged pore structure when compared with the silk meringue.

Many of the most preferred hot-pressed silk meringues include glycerol, as its inclusion provides an impressive malleability, thereby allowing compression with the retention of the general material and pore structure of the silk meringue. Specifically, in some cases where glycerol is present, the hot-pressed silk meringue is a mysilkium material. Specifically, in some cases where glycerol is present, the hot-pressed silk meringue can be or can form a part of (e.g., one or two layers adhered to a fabric substrate) a silk leather. In an embodiment, meringues disclosed herein can be used as an alternative to polyurethane foams employed for artificial leathers.

Without wishing to be bound by any particular theory, it is believed that the most effective bonding between layers of material disclosed herein involves interlinking with mechanical structures (e.g., fibers in a woven fabric)

In some cases, the hot-pressed silk meringue (or other material format disclosed herein) can be interlinked with fabric, but in other cases the hot-pressed silk meringue is interlinked with a metal mesh, a conducting mesh, an electronic component, an active interface, an insulating interface, or a simple coating.

Given that the hot-pressed silk meringue is a downstream product from the silk meringue, the hot-pressed silk meringue can include any component or feature of the silk meringue, unless the context clearly dictates otherwise (e.g., the porosity is reduced in the hot-pressed silk meringue). Similarly, as all of the hot-pressed silk meringue, the silk meringue, and the whipped silk cream derive from upstream entities, the hot-pressed silk meringue can include any component or feature of the silk meringue, the whipped silk cream, or the liquid composition, unless the context clearly dictates otherwise.

The hot-pressed silk meringue has a water content that can be tailored for specific uses. The hot-pressed silk meringue has a water content of between 2% and 50%.

### Methods of Making

The present disclosure provides methods of making the various compositions, materials, and articles described herein.

In one case, the present disclosure provides a method of making a composition, such as a whipped silk cream. A method of making a whipped silk cream can include whipping a liquid comprising silk fibroin and xanthan gum (and, in many cases, plasticizer or functionalizing agent) for a predetermined whipping time to form the whipped silk cream. The predetermined whipping time can be between 5 minutes and 30 minutes, including but not limited to, at least 5 minutes, at least 6 minutes, at least 7 minutes, at least 8 minutes, at least 9 minutes, or at least 10 minutes and at most 30 minutes, at most 25 minutes, at most 20 minutes, at most 15 minutes, or at most 10 minutes.

The whipping can be achieved with mechanical agitation action that is associated with the integration of air, as would typically be understood from the context of food preparation involving various dairy and egg products, among other things. In some cases, the whipping is performed with a whisk. The whipping can optionally be performed manually, though there may be advantages to automated whipping, such as increased speed, endurance, and the like. The whipping can be performed using conventional whipping equipment, such as a stand mixer. The whisk itself can be composed of metal or the whisk can be non-metal (or a metal whisk coated with a non-metal material, in some cases). The whipping can be done within a mixing bowl, such as a metal mixing bowl, for example a stainless steel mixing bowl.

In one specific case, the whipping involves whipping of a heterogenous solution, in which water and glycerol are the liquid phase and the silk fibroin and xanthan gum are in powder form.

In some cases, the temperature of the whipping is maintained at room temperature or lower, including refrigerated temperatures of between 1° C. and 25° C.

In another case, the present disclosure provides a method of making a composition, such as a silk meringue. A method of making a silk meringue can include baking the whipped silk cream (optionally along with any method steps involved in preparing the whipped silk cream itself) at a temperature of between 30° C. and 150° C. or between 50° C. and 80° C. for a length of time of between 2 hours and 24 hours to form the silk meringue. In an example, the baking is performed at a temperature of between 40° C. and 150° C. for a length of time of between 5 minutes and 24 hours.

In some cases, the present disclosure provides a method of making an article, such as a compressed silk meringue. A method of making a compressed silk meringue can include compressing the silk meringue with a force of between 0.25 MPa and 25 MPa for a length of time of between 15 minutes and 6 hours. In some cases, the compressing can be heat-compressing that is performed at an elevated temperature of between 80° C. and 200° C. In some cases, the compressing can be performed with a calendar press. A method of making a compressed silk meringue can include compressing the silk meringue with a force of between 0.25 MPa and 25 MPa for a length of time of between 5 minutes and 6 hours.

In some cases, the methods disclosed herein can further include embossing an article. The embossed article, such as an embossed silk leather, can have an appearance strikingly similar to embossed conventional leather.

The properties of silk creams, silk meringues, silk foams (baked silk creams), and articles of manufacture made from or including the silk creams, meringues, and or foams can be tuned by selection of weight ratios of starting materials, identity of starting materials, conditions of making the creams, meringues, and foams, and the like.

In some aspects, a method of making a whipped silk cream having a desired whipped silk cream morphology, a silk meringue having a desired silk meringue morphology, a compressed silk meringue having a desired compressed silk meringue morphology, or a hot-pressed silk meringue having a desired hot-pressed silk meringue morphology may include selecting a silk concentration, a silk molecular weight distribution, a polysaccharide species, a polysaccharide concentration, a plasticizer species, a plasticizer concentration, a whipping speed, optionally a silk meringue baking temperature, optionally a compressed silk meringue compressing force, optionally a hot-pressed silk meringue hot-pressing force and temperature. The method may further include making the whipped silk cream, the silk meringue, the compressed silk meringue, or the hot-pressed silk meringue using the silk concentration, the silk molecular weight distribution, the polysaccharide species, the polysaccharide concentration, the plasticizer species, the plasticizer concentration, the whipping speed, optionally the silk meringue baking temperature, optionally the compressed silk meringue compressing force, and optionally the hot-pressed silk meringue hot-pressing force and temperature.

For example, the density, water content, and syneresis of the creams can be varied based on the selected weight ratio of silk fibroin, polysaccharide, and plasticizer in the composition. For example, compositions to be used in food or pharmaceutical industries may be tuned to exhibit low syneresis, while compositions to be used in dried foams where mechanical stability is favored may be tuned to exhibit a higher amount of syneresis. In certain aspects, the polysaccharide may have the greatest impact on overall density, the silk fibroin may have the greatest impact on water content, and the plasticizer may have the greatest impact on syneresis. The discovery of these differential impacts enables the tuning of the composition in accordance with the desired application or the desired performance.

In another example, the properties of density, firmness, and overrun of the cream may vary with whipping time. For example, depending on the application, greater or less firmness may be desired, and whipping time may be used to tune the cream for the desired application.

In another example, the properties of a cream's density or overrun may be tuned by selection of a particular plasticizer species. In an aspect, higher numbers of —OH groups of a plasticizer, such as glycerol and 1-3-6 hexanetriol, reduces the whipping time to overrun plateau by efficiently facilitating a hydrogen bonding network between the silk fibroin and the polysaccharide. In another aspect, the distance of —OH groups in a plasticizer influences the air capacity of the foam—plasticizers such as diols (e.g., 1,2 and 1,5 pentanediol) exhibit slower cream growth with a significantly higher overrun when the OH groups are at a greater distance.

In some aspects, the properties of silk foams made from creams and/or meringues may also be tuned via the composition ratio. For example, varying the amount of plasticizer may have an affect on the foam's compressive strength and/or yield point. In another example, varying the polysaccharide species or combination of polysaccharides may be useful in tuning the foam for certain applications. For example, xanthan gum may be useful for applications where the foam is compressed (e.g., silk leather) where alginate may be more useful for foams being used in their expanded state.

In some aspects, the properties of silk foams made from creams and/or meringues may also be impacted by additives, such as borate ions which improve mechanical properties while providing flame retardant and anti-fungal properties.

In some aspects, varying the whipping time may have an affect on the resultant dried foam, such as due to the distribution of bubble sizes or the open/closed cell morphology. For example, longer whipping times may result in denser foams. As whipping time increases, cell structure may transition from a closed cell structure to an open cell structure. Variation in internal structure of foams may have an impact on performance in certain applications, such as in leather applications.

### Articles of Manufacture

The present disclosure provides articles of manufactures that include or are made from one or more of the liquid compositions, whipped silk creams, silk meringues, compressed silk meringues, or hot-pressed silk meringues disclosed herein.

### Silk Leather

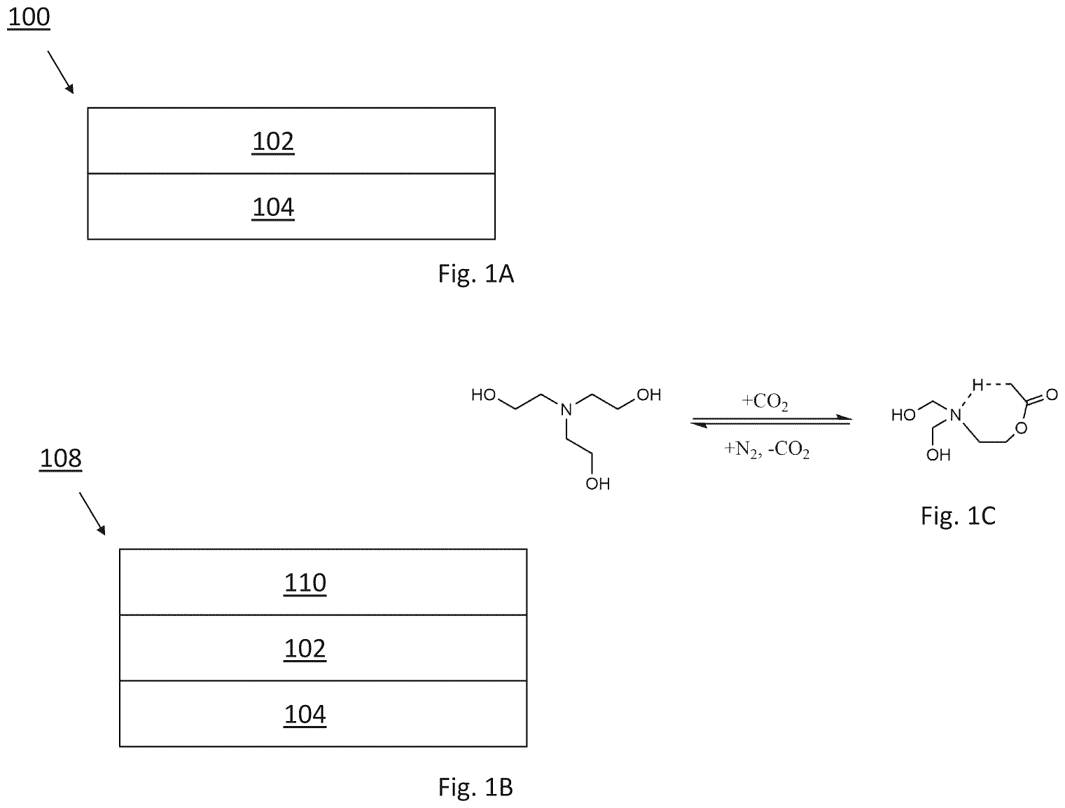

The present disclosure provides a silk leather. The silk leather is a layered structure comprising a first fabric layer and a second material layer disposed adjacent to the first fabric layer. The second material layer comprises the compressed silk meringue or the hot-pressed silk meringue disclosed herein. The silk leather optionally includes a third material layer disposed adjacent to the first fabric layer on a surface opposing the surface to which the second material layer is adjacent. The third material layer comprises the compressed silk meringue or the hot-pressed silk meringue disclosed herein. In effect, the compressed or hot-pressed silk meringues form a “sandwich” around the first fabric layer. Referring to FIG. 1, the silk leather 100 comprises a first fabric layer 102 and a second material layer 104 disposed adjacent to the first fabric layer 102 (See FIG. 1A). In aspects, a silk leather 108 optionally includes a third material layer 110 disposed adjacent to the first fabric layer 102 on a surface opposing the surface to which the second material layer 104 is adjacent (See FIG. 1B).

The first fabric layer can be composed of cotton fabric, including but not limited to, cotton jersey, cotton canvas, and the like; silk fabric; synthetic fabric, including but not limited to, polyester fabric, rayon fabric, nylon fabric, and the like; linen; organza; the like; and combinations thereof.

In the aspects described herein, a unitary portion of silk leather may homogenously exhibit a property or functionality throughout the silk leather, or it may heterogeneously include various portions exhibiting one or more properties or functionalities (e.g., conductive, magnetic, colored, scented, tanned, patterned/embossed/textured, sensing/responsive (e.g., gas, humidity, thermochromic, pH), absorbing, thermal insulator, biological scaffold, electronics, semiconductor device-embedded, haptic, impact-resistant, heat-resistant, cold-resistant, or the like).

The silk leather can be colored to mimic non-colored natural leather. In some cases, the silk leather can be colored to mimic artificially colored natural leather. In one specific case, an artificial leather color can be produced by polymerizing phloroglucinol.

In some cases, using very cold water in making the whipped silk cream from which a silk leather is made can cause a thin “skin” to form on the surface, which can provide a wrinkled texture that mimics the texture of leather.

The present disclosure provides a thermal insulator. The thermal insulator comprises, consists essentially of, or consists of the silk meringue disclosed herein. The thermal insulator can be made by any of the methods disclosed herein. In aspects, thermally insulating silk leather may have a thermochromic reporting property throughout a bulk interior volume.

The present disclosure provides a sorbent and/or gas sensing material. The sorbent and/or gas sensing material can comprise, consist essentially of, or consist of the silk meringue described herein. The sorbent and/or gas sensing material can further include a sensing agent that undergoes a measurable change upon exposure to a gas of interest. The sorbent and/or gas sensing material can include as a sensing agent a dye that changes color upon exposure to the gas of interest. The sorbent and/or gas-sensing material can be made by any of the methods disclosed herein.

The present disclosure provides a biological scaffold. In general, the biological scaffold is intended for the purpose of receiving a population of cells for one or more of growth, proliferation, differentiation, carbon dioxide capture, biomineralization, biosynthesis, fermentation, the like, and combinations thereof.

In one specific case, the biological scaffolds disclosed herein are particularly excellent for algae growth. Specifically, both marine and freshwater algae were seeded and successfully grown on scaffolds for at least a month at 90% relative humidity and under adequate lighting conditions.

In one particular case, the present disclosure provides a mysilkium material with material properties that closely mimic the material properties of mycelium. The mysilkium material can be used in applications where mycelium is currently used.

### Conductive Silk Leather

The present disclosure provides a conductive silk leather. Conductive silk leathers may be used to power embedded lights, for central processing for distributed sensors, and as a component of circuitry. In aspects, a unitary portion of silk leather may include portions that are conductive and portions that are not conductive.

Conductive silk leathers can be used with any other product or article of manufacture described herein. For example, the conductive silk leather can further include sorbent and/or gas sensing material, such as for example to provide a safety garment for a hazardous environment with embedded, powered lights and gas sensing capabilities. In another example, conductive silk leathers can be used with magnetic silk leathers, described elsewhere herein, to form a multi-functional item with magnetic and conductive properties. In some aspects, the conductive silk leather may also include materials, such as magnetic particles or chromium oxide, to render it both conductive and magnetic. Conductive silk leathers can be used with or include a thermal insulator to provide items with conductive and thermal insulation properties.

The conductive silk leather can comprise, consist essentially of, or consist of any of the silk meringues described herein. In aspects, at least one of a plurality of graphite flakes or a graphene powder is distributed within the compressed silk meringue or the hot-pressed silk meringue of the conductive silk leather. The graphite flakes or graphene powder may be added before or during the whipping process, or after whipping. In one example, graphite flakes or graphene powder are added when the whipped silk cream reaches a particular overrun value. The graphite flakes or graphene powder may be added before or during meringue formation or may be added before or during compression or hot-pressing the meringue.

In some aspects, the conductive silk leather includes a conductive ink. For example, the conductive ink is printed on a surface of the compressed silk meringue or the hot-pressed silk meringue. In another example, the conductive ink is printed between layers of the compressed silk meringue or the hot-pressed silk meringue or printed and subsequently embedded within the compressed silk meringue or the hot-pressed silk meringue. In yet another example, the conductive ink is printed between the compressed silk meringue or the hot-pressed silk meringue and a fabric layer. The conductivity is patterned into an electronic circuit. The resistivity of the conductive silk leather is at most 1kΩ, at most 0.7kΩ, at most 0.5kΩ, or at most 0.1kΩ.

For the avoidance of doubt, the conductive silk leathers described herein can wholly include or include features of the silk leathers disclosed elsewhere here. Similarly, the conductive silk leathers described herein can be made from, comprise, wholly include, or include features from the liquid compositions, whipped silk creams, silk meringues, compressed silk meringues, hot-pressed silk meringues, or methods disclosed elsewhere herein.

### Magnetic Silk Leather

The present disclosure provides a magnetic silk leather. The magnetic silk leather can comprise, consist essentially of, or consist of any of the silk meringues described herein. In aspects, at least one of a plurality of magnetic particles or a plurality of chromium oxide particles is distributed within the compressed silk meringue or the hot-pressed silk meringue. The plurality of magnetic particles or plurality of chromium oxide particles may be added before or during the whipping process, or after whipping. In one example, plurality of magnetic particles or plurality of chromium oxide particles are added when the whipped silk cream reaches a particular overrun value. The plurality of magnetic particles or plurality of chromium oxide particles may be added before or during meringue formation or may be added before or during compression or hot-pressing the meringue. In an aspect, a magnetic field may be applied at any point during the processing of the liquid composition to a silk whipped cream and silk meringues. Magnetic silk leathers can block RFID signals or be a building block for products such as robots, games, home organizing/décor, or the like.

The magnetic silk leather can be tailored to have a specific polarity at a surface of the magnetic silk leather. In some cases, the polarity is North. In some cases, the polarity is South.

In some cases, the magnetic material may be manipulated prior to curing, such that a specific magnetic configuration is locked into the magnetic silk leather. Conceptually, applications involving ferrofluids such as generating different patterns could be applied to creation of magnetic silk foams/meringues/leathers. In general, a magnetic field could be applied at any step of the methods disclosed herein with the intention of manipulating magnetic particles located within one or more of the compositions disclosed herein.

In some cases, the magnetic silk leather can be used as an external surface for a robot (e.g., a “robot skin”).

For the avoidance of doubt, the magnetic silk leathers described herein can wholly include or include features of the silk leathers disclosed elsewhere here. Similarly, the magnetic silk leathers described herein can be made from, comprise, wholly include, or include features from the liquid compositions, whipped silk creams, silk meringues, compressed silk meringues, hot-pressed silk meringues, or methods disclosed elsewhere herein.

### Scented Silk Leather

The present disclosure provides a scented silk leather. The scented silk leather can comprise, consist essentially of, or consist of any of the silk meringues described herein. In aspects, a plurality of aromatic compounds are distributed throughout the compressed silk meringue or the hot-pressed silk meringue of the scented silk leather.

The aromatic compounds may include emulsions, oils, alcohols, scented powder, or the like. The character of the aromatic compounds can impact the processing and the methods disclosed herein may afford different processing requirements. Both oil and alcohols can be freely incorporated into creams during the whipping, especially if the fragrance is concentrated (e.g., such as if the oil/alcohol is less than 5% of total volume). For adsorbing the fragrance on a dry material (e.g., meringue or leather), both oils and alcohols may be useful, but the permeability of the material would be controlled by the nature and content of the plasticizer. For example, foams with no glycerol (e.g., pure silk fibroin and xanthan gum) display a higher oil sorption capacity.

Applying an additional volume of the aromatic compound to a surface of the scented silk leather, such as by spraying, wiping, submerging, or other application method, may at least partly recharge the scented silk leather, thereby extending the lifetime of aroma release. In some cases, alcohol-based aroma compounds can be particularly effective at recharging scented silk leathers.

The aromatic compounds may be added before or during the whipping process, or after whipping. In one example, aromatic compounds are added when the whipped silk cream reaches a particular overrun value. The aromatic compounds may be added before or during meringue formation or may be added before or during compression or hot-pressing the meringue.

The silk foam can exhibit heterogeneous domains of scent such that scent intensity can be distributed based on foam particles. Through compression, gradients of scent may be generated. In one aspect, the scented silk leather exhibits pressure-sensitive aroma release.

The ability of a silk leather to become scented, maintain scent, release/dispense/disperse scent, and/or recharge scent may depend on factors such as the components of the liquid composition, the density, the water content, the presence of other agents/additives in the silk leather, or the like. Likewise, the release profile of scents may depend on similar factors.

For the avoidance of doubt, the scented silk leathers described herein can wholly include or include features of the silk leathers disclosed elsewhere here. Similarly, the scented silk leathers described herein can be made from, comprise, wholly include, or include features from the liquid compositions, whipped silk creams, silk meringues, compressed silk meringues, hot-pressed silk meringues, or methods disclosed elsewhere herein.

### pH Responsive Silk Leather

The present disclosure provides a pH-responsive silk leather. The pH-responsive silk leather can comprise, consist essentially of, or consist of any of the silk meringues described herein. In aspects, a pH-responsive chemical is distributed throughout the compressed silk meringue or the hot-pressed silk meringue of the pH-responsive silk leather. The pH-responsive chemical may be added before or during the whipping process, or after whipping. In one example, a pH-responsive chemical is added when the whipped silk cream reaches a particular overrun value. The pH-responsive chemical may be added before or during meringue formation or may be added before or during compression or hot-pressing the meringue. pH-responsive silk leather may be useful in worn garments such as to alert a wearer of certain ambient or precipitating pollutants.

For the avoidance of doubt, the pH-responsive silk leathers described herein can wholly include or include features of the silk leathers disclosed elsewhere here. Similarly, the pH-responsive silk leathers described herein can be made from, comprise, wholly include, or include features from the liquid compositions, whipped silk creams, silk meringues, compressed silk meringues, hot-pressed silk meringues, or methods disclosed elsewhere herein.

### Humidity Sensing Silk Leather

The present disclosure provides a humidity sensing silk leather. The humidity sensing silk leather can comprise, consist essentially of, or consist of any of the silk meringues described herein. In aspects, a pH responsive chemical (e.g., o-cresolphthalein) and a pH altering agent (e.g., sodium carbonate) are distributed throughout the compressed silk meringue or the hot-pressed silk meringue of the humidity sensing silk leather. In aspects, measurable amounts of humidity solubilize at least a portion of the pH altering agent, thereby lowering the pH, thereby providing a measurable report of humidity. At least one of the pH responsive chemical or the pH altering agent may be added before or during the whipping process, or after whipping. In one example, at least one of the pH responsive chemical or the pH altering agent are added when the whipped silk cream reaches a particular overrun value. At least one of the pH responsive chemical or the pH altering agent may be added before or during meringue formation or may be added before or during compression or hot-pressing the meringue. Humidity sensing silk leathers may be useful, for example, as a component of a humidor box.

For the avoidance of doubt, the humidity sensing silk leathers described herein can wholly include or include features of the silk leathers disclosed elsewhere here. Similarly, the humidity sensing silk leathers described herein can be made from, comprise, wholly include, or include features from the liquid compositions, whipped silk creams, silk meringues, compressed silk meringues, hot-pressed silk meringues, or methods disclosed elsewhere herein.

### Patterned Silk Leather

The present disclosure provides a patterned silk leather. The patterned silk leather can comprise, consist essentially of, or consist of any of the silk meringues described herein. Patterned silk leathers may exhibit any pattern or texture, such as for example to mimic reptile skin in appearance and/or feeling. Certain patterns or textures may provide an aesthetic quality and/or a function/property to silk leather, such as improved grip, anti-bacterial, anti-fouling, water-repellent, waterproof, dust-repellent, or the like. The dimensions of the patterns may be nano-, micro-, or macro-scale.

Examples of suitable surface patterns include, but are not limited to: leather mimicking patterns, which mimics a variety of different leathers, including alligator leather, crocodile leather, snake leather, cow leather, stingray leather, ostrich leather; tessellating patterns; optically-active patterns, such as diffraction patterns; plant patterns, geometric patterns, letters and numbers, or the like.

For the avoidance of doubt, the patterned silk leathers described herein can wholly include or include features of the silk leathers disclosed elsewhere here. Similarly, the patterned silk leathers described herein can be made from, comprise, wholly include, or include features from the liquid compositions, whipped silk creams, silk meringues, compressed silk meringues, hot-pressed silk meringues, or methods disclosed elsewhere herein.

### Electronic Embedded Silk Leather

The present disclosure provides an electronic silk leather having an electronic component embedded therein. The electronic silk leather can comprise, consist essentially of, or consist of any of the silk meringues described herein. In aspects, the electronic component or a second electronic component is embedded between the silk layer and the fabric layer in the electronic silk leather. In aspects, the electronic component or a third electronic component is embedded within the silk layer in the electronic silk leather. In aspects, the electronic silk leather further comprises a power supply coupled to the electronic component. For example, the power supply may be a rechargeable battery, a wired disposable battery holder, fiber-shaped solar cells (e.g., perovskite solar cells), or a combination thereof. In aspects, the electronic component comprises an RFID tag. In aspects, the electronic component comprises wiring. The present disclosure also provides a silk cream or silk meringue having electronics distributed throughout. In aspects, the silk cream or silk meringue may be a variable density filler material having electronic functionalization. In embodiments, electronic components may include light emitting devices such as LEDs or electroluminescent wires. Such components may be useful in fashion and design applications as well as for powering sensing applications.

For the avoidance of doubt, the electronic silk leathers described herein can wholly include or include features of the silk leathers disclosed elsewhere here. Similarly, the electronic silk leathers described herein can be made from, comprise, wholly include, or include features from the liquid compositions, whipped silk creams, silk meringues, compressed silk meringues, hot-pressed silk meringues, or methods disclosed elsewhere herein.

### Semiconductor Device Embedded Silk Leather

The present disclosure provides a semiconductor device-embedded silk leather having a semiconductor device embedded therein. The semiconductor device-embedded silk leather can comprise, consist essentially of, or consist of any of the silk meringues described herein. The semiconductor device or a second semiconductor device may be embedded at a surface of the compressed silk meringue or the hot-pressed silk meringue. The semiconductor device or a third semiconductor device may be embedded within the compressed silk meringue or the hot-pressed silk meringue. The semiconductor device or a fourth semiconductor device may be embedded between the first fabric layer and the compressed silk meringue or the hot-pressed silk meringue. In aspects, the semiconductor device-embedded silk further comprises a power supply, as described elsewhere herein, coupled to the semiconductor devices. In aspects, the semiconductor device may be in communication with one or more electronic components in the silk leather.

For the avoidance of doubt, the semiconductor device-embedded silk leathers described herein can wholly include or include features of the silk leathers disclosed elsewhere here. Similarly, the semiconductor device-embedded silk leathers described herein can be made from, comprise, wholly include, or include features from the liquid compositions, whipped silk creams, silk meringues, compressed silk meringues, hot-pressed silk meringues, or methods disclosed elsewhere herein.

### Haptic Silk Leather

The present disclosure provides a haptic silk leather having a haptic switch embedded therein. The haptic silk leather can comprise, consist essentially of, or consist of any of the silk meringues described herein. The haptic switch or a second haptic switch may be embedded at a surface of the silk leather. The haptic switch or a third haptic switch may be embedded within the compressed silk meringue or the hot-pressed silk meringue. The haptic switch or a fourth haptic switch may be embedded between the first fabric layer and the compressed silk meringue or the hot-pressed silk meringue. In aspects, the haptic silk further comprises a power supply, as described elsewhere herein, coupled to the haptic switch. In aspects, the haptic switch may be in communication with one or more electronic components or semiconductor devices in the silk leather.

For the avoidance of doubt, the haptic silk leathers described herein can wholly include or include features of the silk leathers disclosed elsewhere here. Similarly, the haptic silk leathers described herein can be made from, comprise, wholly include, or include features from the liquid compositions, whipped silk creams, silk meringues, compressed silk meringues, hot-pressed silk meringues, or methods disclosed elsewhere herein.

### Tanned Silk Leather

The present disclosure provides a tanned silk leather. The tanned silk leather can comprise, consist essentially of, or consist of any of the silk meringues described herein. The tanned silk leather may have a bulk volume of a compressed silk meringue or hot-compressed silk meringue and a surface layer of the compressed silk meringue or hot-pressed silk meringue. The surface layer may be formed from the same chemical composition as the bulk volume but includes at least one differing structural, mechanical, or chemical feature relative to the bulk volume. In an aspect, the surface layer includes a dye.

In an aspect, the surface layer comprises a material, such as a precursor material, that may mimic tanning products (e.g., absorb aniline/dyes). In aspects, the surface layer may be modified/re-shaped relative to the bulk volume, such as by surface patterning, to provide a material difference to the surface layer. Silk leathers subjected to processes akin to tanning (e.g., chemical treatment with aniline) may result in a relatively stiff material which may, for example, be relatively more processable for fashion industries.

For the avoidance of doubt, the tanned silk leathers described herein can wholly include or include features of the silk leathers disclosed elsewhere here. Similarly, the tanned silk leathers described herein can be made from, comprise, wholly include, or include features from the liquid compositions, whipped silk creams, silk meringues, compressed silk meringues, hot-pressed silk meringues, or methods disclosed elsewhere herein.

### Fiber Fortified Silk Cream/Meringue

The present disclosure provides a silk cream or silk meringue having fibers distributed throughout, such as fibers/textiles described herein. In aspects, woven fabrics may be penetrated with the liquid composition or any of the downstream products described herein.

For the avoidance of doubt, the fiber fortified silk cream/meringue can wholly include or include features of the whipped silk creams or silk meringues disclosed elsewhere herein. Similarly, the fortified creams/meringues described herein can be made from, comprise, wholly include, or include features from the liquid compositions, whipped silk creams, silk meringues, compressed silk meringues, hot-pressed silk meringues, or methods disclosed elsewhere herein.

### Ultra-Lightweight Silk Down Alternative

The present disclosure provides an ultra-lightweight silk down alternative comprising, consisting essentially of, or consisting of any of the silk meringues described herein. The ultra-lightweight silk down alternative is made from a regenerated silk fibroin solution made from a recycled regenerated silk fibroin article or a waste silk fabric. The ultra-lightweight silk down alternative may further include a plurality of heat-reflective particles and/or have a thermochromic reporting capability.

### Impact Distributing Foam

The present disclosure provides an impact-distributing foam comprising a silk meringue. The impact-distributing foam has one or more impact and/or strain sensors positioned within the impact-distributing foam. The one or more impact and/or strain sensors are selected from the group consisting of a PDA sensor, an accelerometer, a piezoelectric sensor, a vibration sensor, a piezoresistive sensor, or a strain gauge sensor. The impact-distributing foam has one or more strain sensors positioned within the impact-distributing foam. The density of the foam may be between 0.1 g/cm³ and 0.2 g/cm³, or between 0.01 g/cm³ and 0.05 g/cm³, r between 0.05 g/cm³ and 0.25 g/cm³. For example, the density of the foam is at least 0.01 g/cm³, at least 0.05 g/cm³, at least 0.1 g/cm³, at least 0.2 g/cm³, or at least 0.25 g/cm³. The density of the foam may be tuned by one or more factors such as the components included in the liquid composition, the weight ratio of one or more components in the liquid composition, the weight % of one or more components in the liquid composition, the amount of whipping time, or the temperature during whipping.

The foams described herein serve two related but distinct functional purposes at the same time. On the one hand, the foam protects an underlying item from impact. On the other hand, the foam protects embedded sensors from the environment. In this way, a variety of different impact sensors may be usable, which otherwise would not be due to stability.

### Protected Item

The present disclosure provides a protected item comprising: an item to be protected; and protective shell comprising a silk meringue, a cured silk meringue, a compressed silk meringue, or a hot-compressed silk meringue. The protective shell is formed by surrounding and contacting the item to be protected with a precursor to the protective shell and curing the precursor to form the protective shell, wherein surrounding comprises fully encapsulating or encapsulating against a surface. For example, the item to be protected may be at least one of an electronics component, an aerospace component, a semiconductor chip, a semiconductor device, or a three-dimensional printed structure.

The protective shell renders the item to be protected resistant to an impact of between 100 Newtons (N) and 25,000N, between 200N and 15,000N, between 500N and 10,000N, or between 1,000N and 5,000N. The level of impact resistance of the protective shell may be tuned by one or more factors such as the components included in the liquid composition, the weight ratio of one or more components in the liquid composition, the weight % of one or more components in the liquid composition, the amount of whipping time, or the temperature during whipping. The level of impact resistance of the protective shell may be tuned to provide a protective shell that is substantially resistant to impact, a protective shell that is moderately resistant to impact (e.g., the protective shell sustains damage while the item does not sustain damage, an outer portion of the protective shell sustains severe damage while an inner portion sustains less damage and the item sustains no damage), or a protective shell is minimally resistant to impact.

The item to be protected is resistant to a temperature of between 80° F. and 500° F., between 100° F. and 400° F., or between 150° F. and 300° F. The item to be protected is resistant to a temperature of between 0° F. and −500° F., between −50° F. and −400° F., or between −150° F. and −300° F.

For certain items, the nature of the material when it is applied may be relevant, such as a water content for applications involving electronics.

In cases where moisture sensitivity is an issue, the whipped silk cream or silk meringue that is applied to the item can have a water content below a threshold value. In cases where low moisture is an issue, the whipped silk cream or silk meringue that is applied to the item can have a water content above a threshold value.

In some cases, the protective shell, the silk meringue, the compressed silk meringue, or the hot-pressed silk meringue can have one or more impact sensors distributed throughout for reporting impacts that exceed a given threshold. In some cases, the impact sensors and/or strain sensors are polydiacetylene (PDA) based sensors.

### Open Cell Silk Foam

The present disclosure provides an open-cell silk foam. The inventors discovered that controlling the whipping during the making of the silk cream can control the nature of the pores that are created in eventual silk foams/meringues. If the whipping falls within a given window of whipping (i.e., exceeds a first whipping threshold, but does not exceed a second whipping threshold), an open-cell pore structure is formed in the whipped silk cream, resulting in an open-cell silk foam/meringue when baked. The open cell foams are generally lighter, more white and reflective, and more permeable to organic solvents. In some embodiments, open cell foams may be preferable for fragrance release or other applications that include loading of the baked foam. Open cell foams may be useful for sensor applications where sensors have to interact with the environment, such as gas, humidity or pH sensors.

### Closed Cell Silk Foam

The present disclosure provides a closed-cell silk foam. The inventors discovered that controlling the whipping during the making of the silk cream can control the nature of the pores that are created in eventual silk foams/meringues. If the whipping falls outside a given window of whipping (i.e., does not exceed a first whipping threshold or does exceed a second, higher whipping threshold), a closed-cell pore structure is formed in the whipped silk cream, resulting in a closed-cell silk foam/meringue when baked. A closed cell morphology may be useful for applications in which an element is added to the foam during whipping and it is desired to preserve the element (e.g., living organisms or mechanical/thermal sensors).

It should be appreciated that there are a host of end uses for the materials described herein. Examples include, but are not limited to, bio-responsive seating, body monitors, leather bracelets as health indicators, enzymatic bracelets, scented bracelets, sensing sofas, fluorescent foams, microelectronic heat dissipators, fuel cell electrode matrices, food flavoring, water purifiers, pool purifiers, ocean oil capture, an ultralight insulator alternative (e.g., alternative to goose down), a leather-like impact monitor, gas sensing personal protective equipment (PPE), and the like.

### EXAMPLES

Example 1. 4 mL of silk solution (boiled 30-180 minutes) at a concentration of 3-7% was mixed with 100 mg of xanthan gum and whipped with a whisk for about ten minutes to obtain a silk cream that can be cooked at 60° C. overnight to obtain a foam. The foam has an irregular porosity, is brittle, has excellent buoyancy, low hydrophilicity and high affinity for organic solvents. The foams can be useful for several applications such as thermal insulators, substrates for gas sensing and biological scaffolds. As proof of the concept, a sponge was used to remove an organic phase (hexane) colored with a dye (oil red O) from a stirred aqueous phase. The sponge was tested as a substrate for gas sensing. In 1 mL chloroform, 2 mg of the pH sensor of bromothymol blue and triethanolamine were dissolved in different ratios to make a sensor for CO₂. The sponges were dried and placed in a desiccator filled with gaseous CO₂. After a short time (e.g., minutes), a color change was appreciated on the sponges whose speed depends on the quantity of triethanolamine (TEA). The reversible CO2 sensing reaction is depicted in FIG. 1C. Furthermore, the sensor is also reversible since, exposed to air, it partially returns to its original color. Different porosity in the SF:XG foam could modulate the sensitivity/responsiveness. Further, the amount of TEA may affect both the speed of color change and its partial recovery.

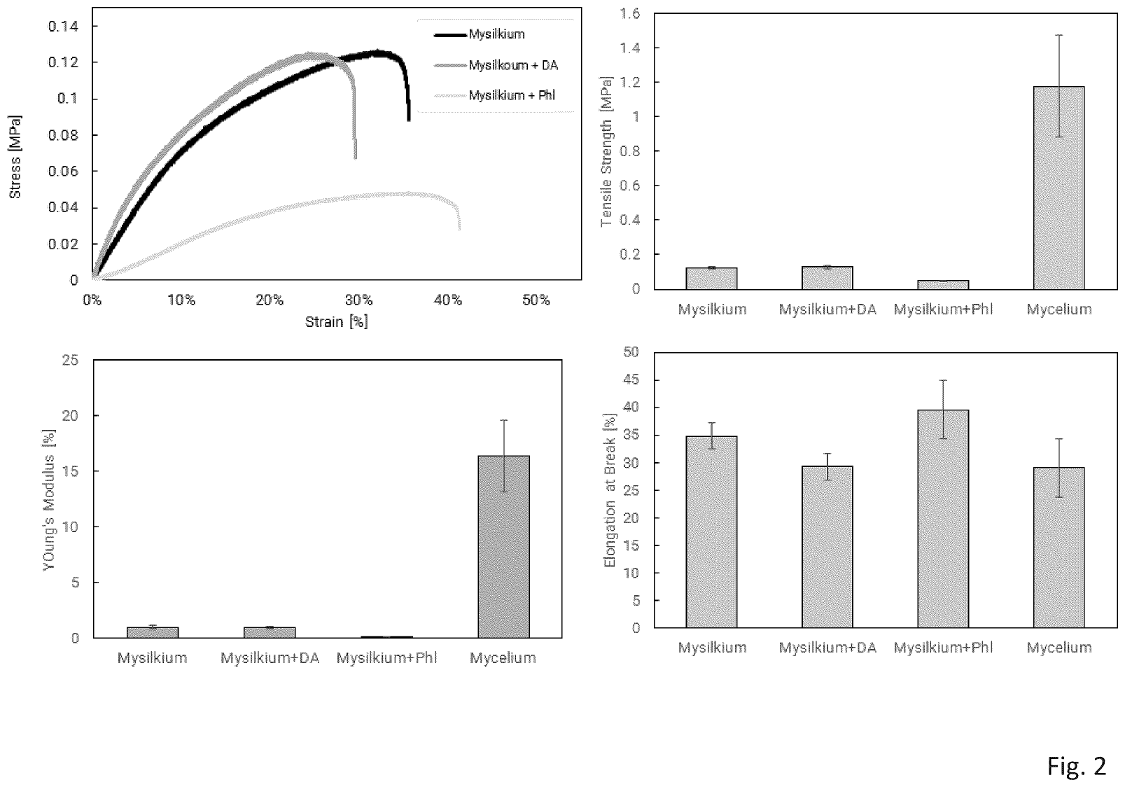

Example 2. Artificial Leathers: By adding glycerol to the composition and following the same process (e.g., a process as detailed in Example 1), the foam obtained after cooking results in a soft material. By hot pressing the foam (0.5-3 Mpa at 150° C. for 30 minutes), a material whose properties are reminiscent of mycelium is obtained, therefore, this material is referred to as “mysilkium”. Under these conditions, the foam also exhibits self-healing capabilities since sheets can be made from isolated foam fragments. The foam was subjected to mechanical tests comparing its properties to those of the mycelium. In some examples, polyphenols were added to the composition, but these do not cause significant changes in mechanical properties using these conditions. Young's modulus and tensile strength of the mycelium are at least an order of magnitude higher than those of the Mysilkium, while the elongation is roughly the same (FIG. 2).

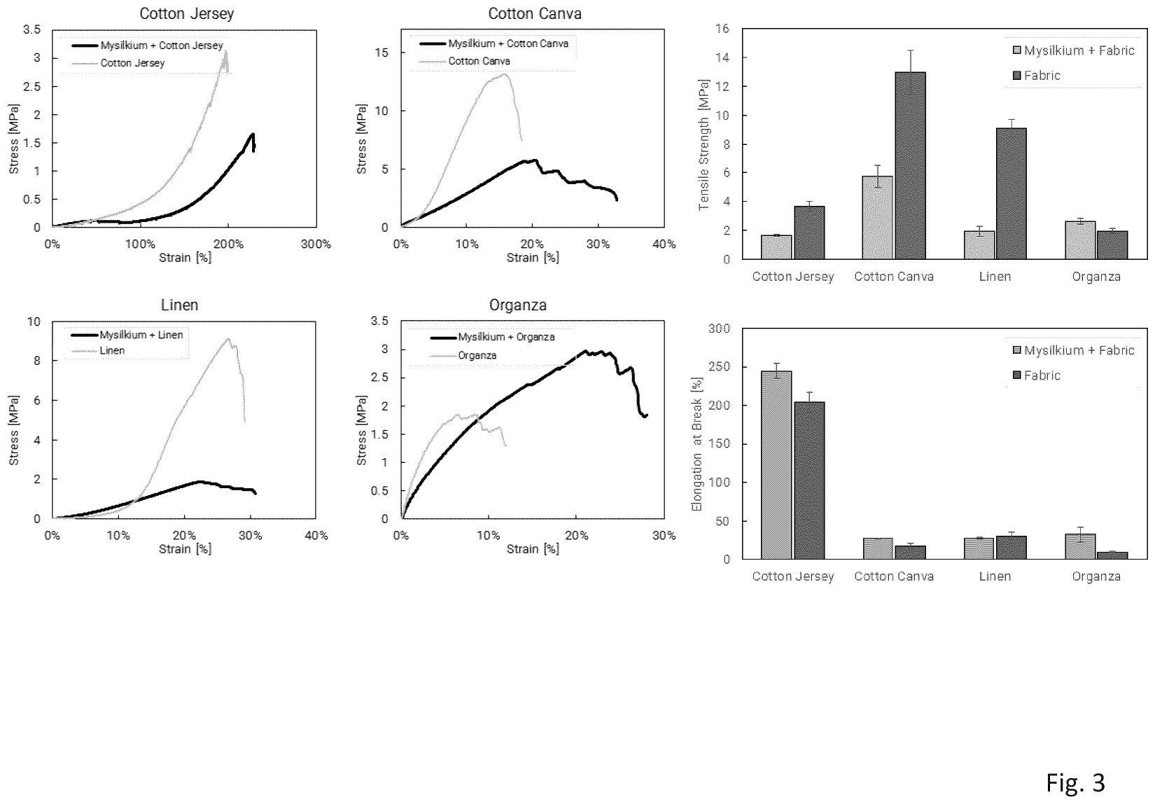

To increase the mechanical properties of the mysilkium, a composite material was made by making “layered cakes” by adding a fabric between two layers of cream before cooking it. The composite foam was then pressed and heated as described previously. With this strategy, materials have been created that aesthetically and to the touch recall the mycelium or artificial skin, but whose mechanical properties are similar to the added fabric/tissue (FIG. 3).

### Example 3. Additional Characterization

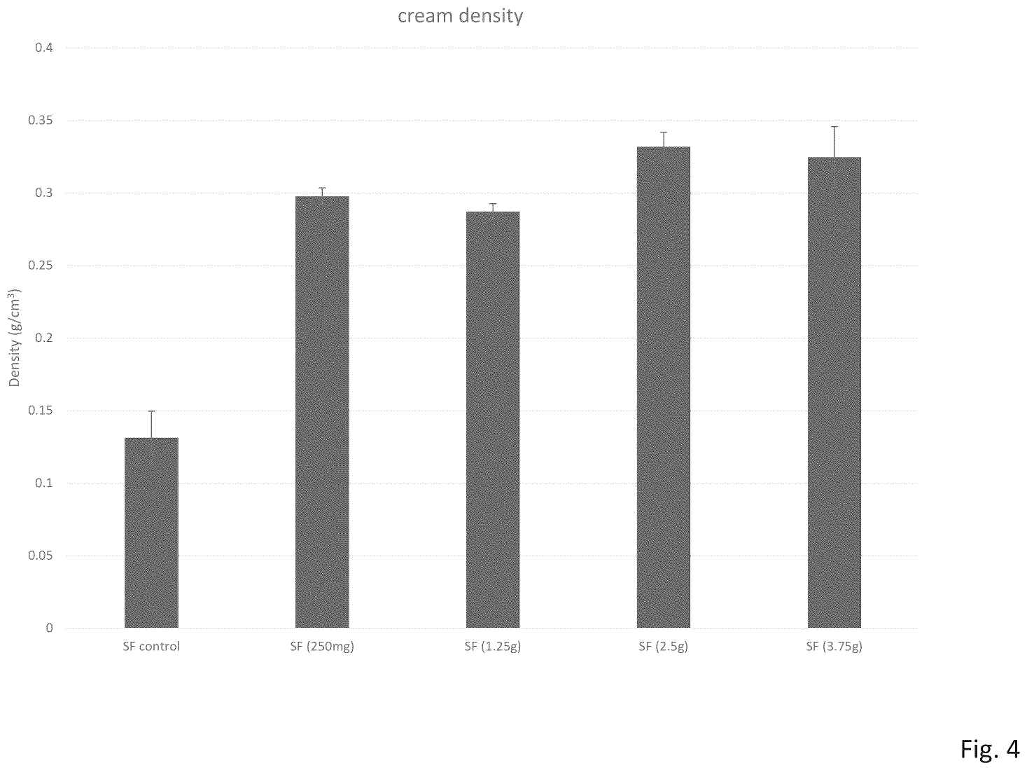

Cream and meringue densities: In 35 mL of bidistilled water, 3 mL of glycerol (gly), 1 gram of xanthan gum (XG) and 250 mg, 1.25 g, 2.5 g and 3.75 of silk fibroin (SF) powder (220 minute boiled) were added. Controls were also prepared with only XG (1 g) and gly (3 mL), and only SF (1 g) and gly (3 mL). All the samples were whipped for 12 minutes to obtain the “cream”. The cream was used to fill 7.5 mL petri dishes and, after weighting the density of the cream was measured (FIG. 4).

Control with XG alone does not whip to cream but forms a sticky slime. Whip only SF and Gly forms a not very consistent cream with a density between 0.1-0.15 g/cm³. The addition of XG increases the density of the creams obtained to around 0.3 g/cm³. Variations in the density of the creams could be more influenced by the amount of water used for whipping or by the whipping time.

The creams were subsequently baked at 60° C. overnight and the weight was measured again. This allowed to estimate the water content of the different creams assuming that the glycerol (boiling point 290° C.) is not lost during the baking process while all the water in the cream is removed.

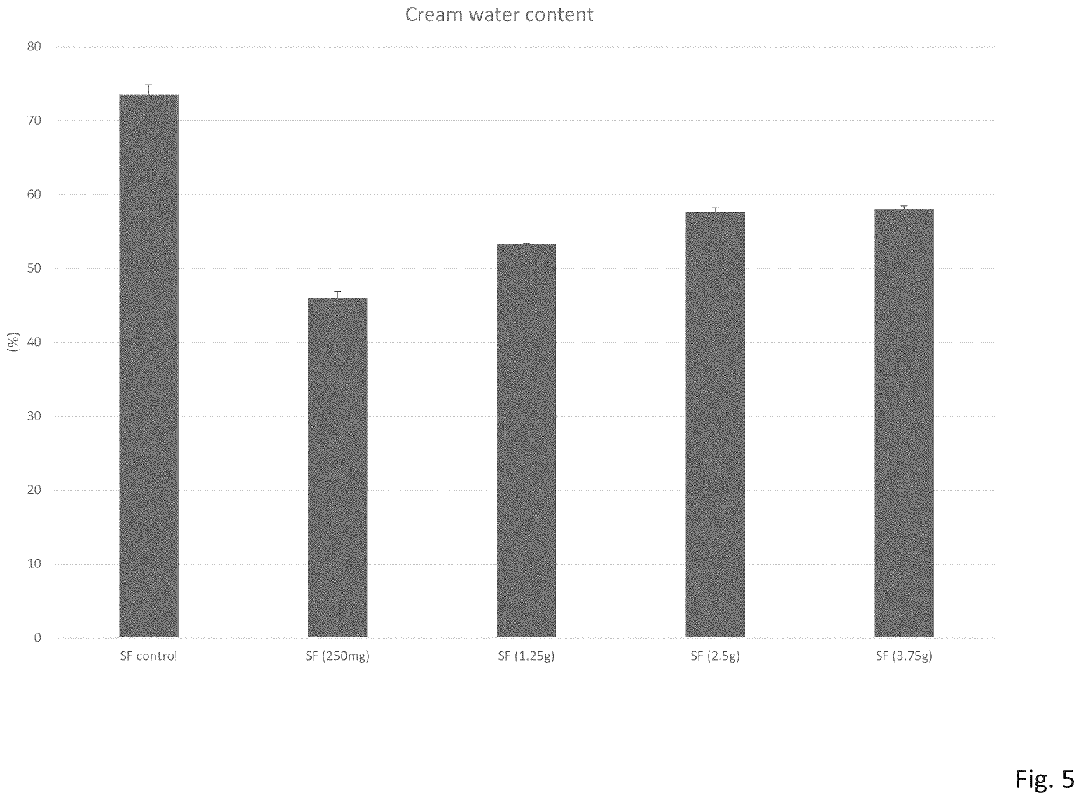

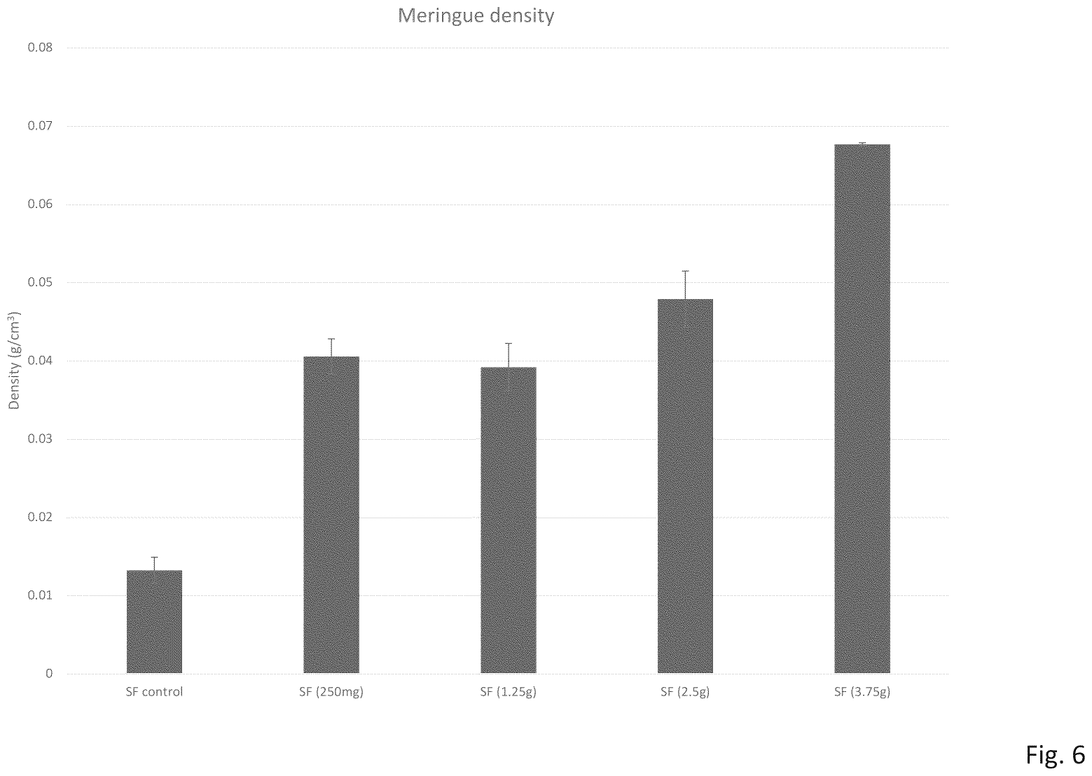

The silk cream has the highest water content (73%) while the addition of XG reduces the water content (46-58%) (FIG. 5). This is likely due to a higher porosity obtained with XG. By increasing the amount of silk compared to XG, the water content of the cream slightly increases.

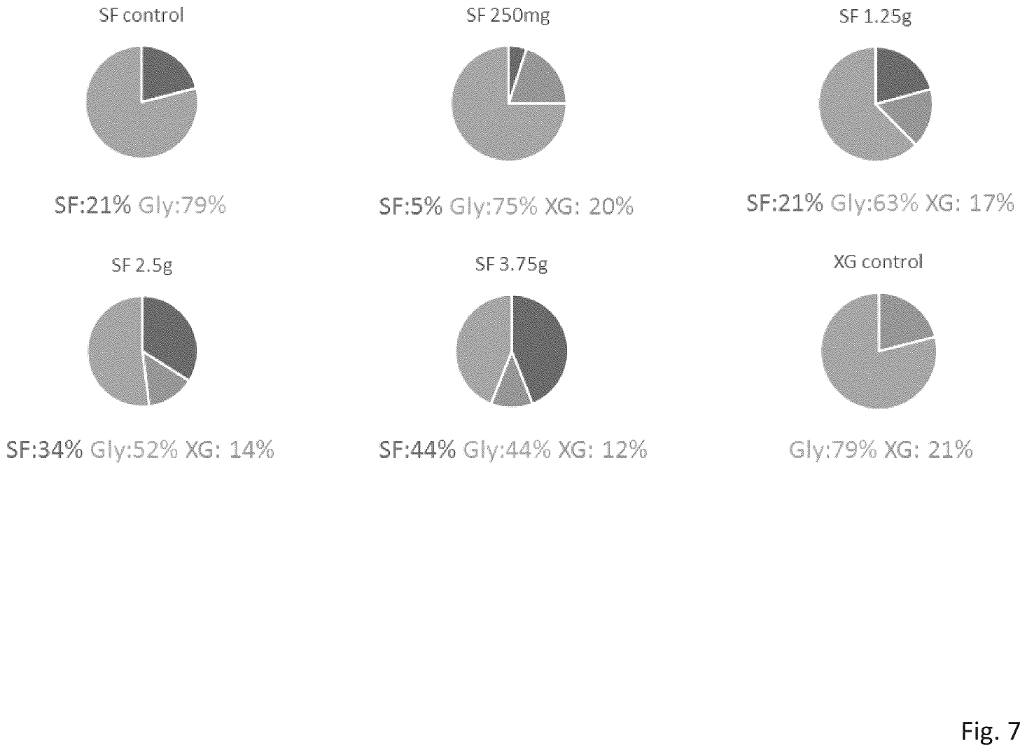

In general, after cooking overnight the cream to meringue, density is reduced by a factor of 10. The density of meringues made of SF is very low (below 0.02 g/cm³). With the combinations of SF and XG the density of the meringues increases around 0.05 g/cm³ for meringues with solid silk contents lower than 34% and reaches 0.06 g/cm³ for meringue with solid SF contents of 44% (see FIG. 7). FIG. 7 shows the solid compositions of the meringues obtained by varying the mass of SF and assuming a total removal of water from the cooking process. From all compositions except the XG control, a cream has been obtained that can be cooked in meringue, and then processed using a hot press (150° C. for 30 minutes) at “mysilkium”.

As the SF content increases, the meringues become gradually stiffer and the same goes for the mysilkium obtained. The tactile perception of the material is comparable to an artificial leather. From a qualitative point of view, the mysilkium obtained with the composition SF21: Gly63: XG17 had the best tactile properties in terms of softness, flexibility, elasticity and homogeneity. Samples with SF 34% and higher content tend to deform over time after being pressed to mysilkium, while with SF<34% content, they remain flat indefinitely.

### Example 4: Alternatives to Silk or Xanthan Gum

The ingredients used to prepare the foams were changed while maintaining a solid ratio of protein20:glycerol60:gum20. Silk Alternatives: Compositions were mixed, baked, and pressed following the same aforementioned process. A “mysilkium” with gelatin, soy protein, casein as protein alternatives and pluronics and PEG (65 kDa) as synthetic alternative was obtained. Gelatin failed to form a cream. Soy protein formed a very liquid film that displayed high water content and stickiness after the hot-pressing process. Casein was the only protein alternative able to form a material similar to mysilkium although the homogeneity, softness and aesthetics were much lower compared to silk. PEG failed to form a cream. With pluronics (65 KDa), a “mysilkium” was formed, but still with homogeneity, softness and aesthetics greatly inferior to those obtained with silk. Pluronic was chosen as an alternative because, like SF, is a polymer organized in an alternation of hydrophobic and hydrophilic domains.

Gum alternatives: Xanthan gum was substituted with pine rosin gum, Arabic gum and tragacanth gum. None of these where suitable to obtain a cream or meringue with similar properties to mysilkium. Guar gum may be used as an alternative to xanthan gum, though it showed inferior performance for the specific applications that were pursued in this disclosure (e.g., to obtain a silk cream that is compressible into other interesting material formats, like silk leather) when compared to xanthan gum.

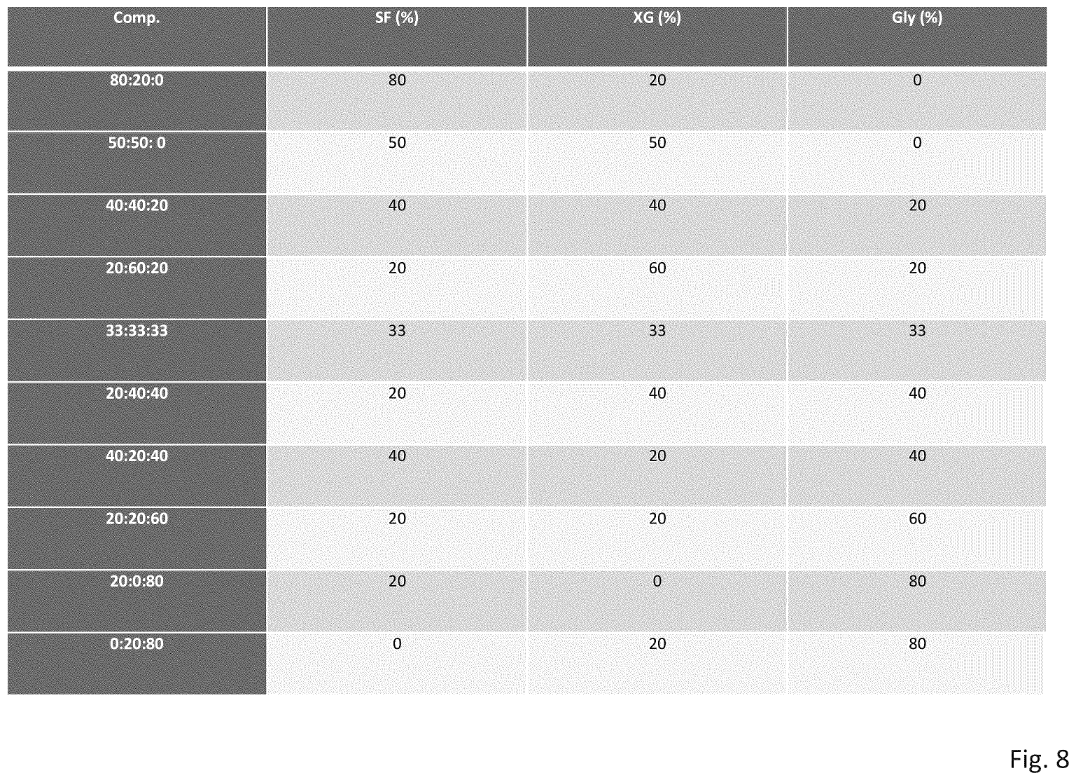

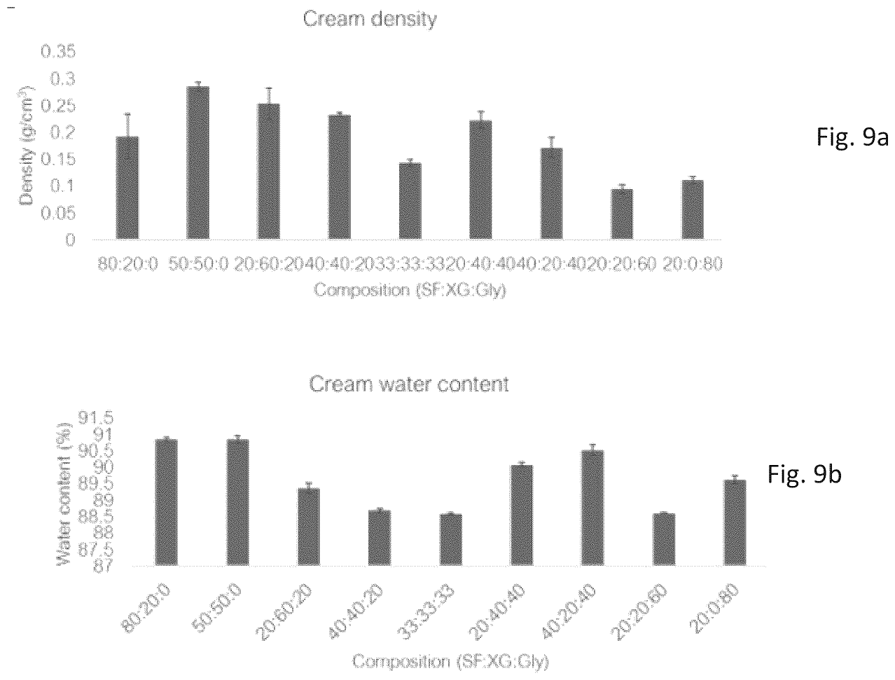

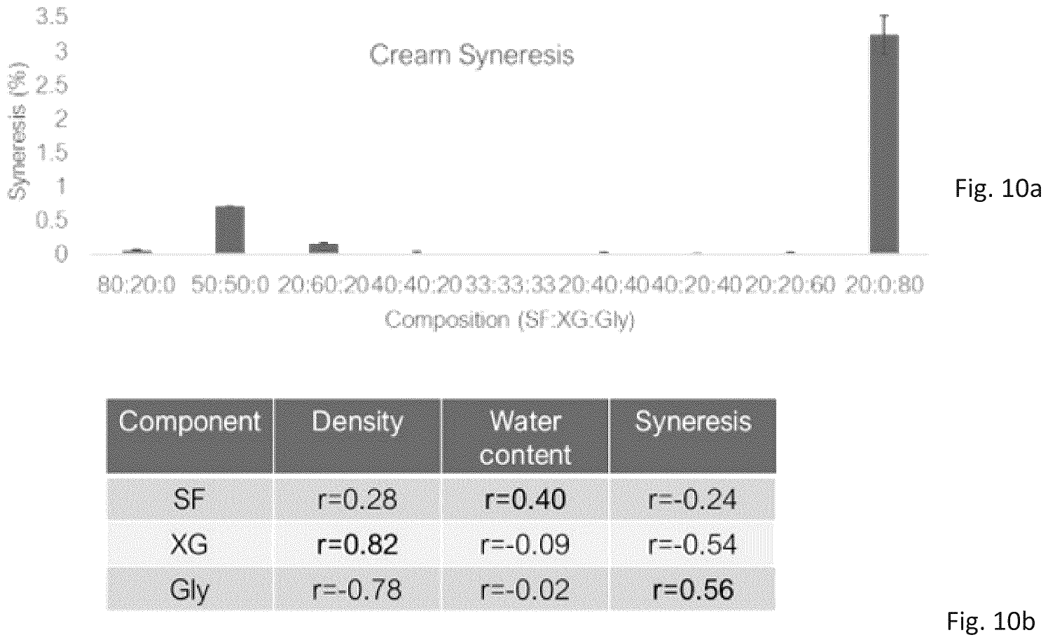

Example 5. Properties related to composition: The density, water content and syneresis of the whipped creams using different compositions with tunability on some parameters has been characterized. FIG. 8 provides data regarding various compositions and the varying weight ratio of SF, Gly, and XG in each composition. FIG. 9a depicts the cream density for the compositions identified in FIG. 8 in g/cm³. FIG. 9b depicts the cream water content for the compositions identified in FIG. 8. FIG. 10a depicts the syneresis (%) for the compositions identified in FIG. 8 and FIG. 10b depicts the correlation of density, water content, and syneresis with the solid content. It is possible to tune the density (important for thermal and mechanical properties, water content (very narrow range, but important for cell cultures) and syneresis which affect cream stability overtime.

Different combinations of SF, XG, and Gly yield varying densities, water contents, and syneresis, indicating that the components have distinct effects on the overall properties of the mixture.

The Pearson correlation coefficient for each component and each variable was calculated, revealing that the content of XG (r=0.82) is the component most closely related to the increase in density that can range between 0.09-0.28 g/cm³. The water content exhibits low variability (88.5%<x<91%) with changes in composition, and SF (r=0.40) is correlated with the water content.

Syneresis refers to the spontaneous expulsion of water from a colloidal system, resulting in the contraction or shrinkage of the material. As shown in FIG. 10a, the water loss for every composition is minimal, being less than 3% for each combination. This low syneresis ensures stability and consistency, making this material particularly interesting for the food or pharmaceutical industries.

Lastly, Gly is the component most strongly correlated with syneresis. While high syneresis negatively impacts foam stability, creams with low glycerol content might initially appear more desirable. However, it's important to note that glycerol content is important for the mechanical performance of the dried foams, as disclosed herein.

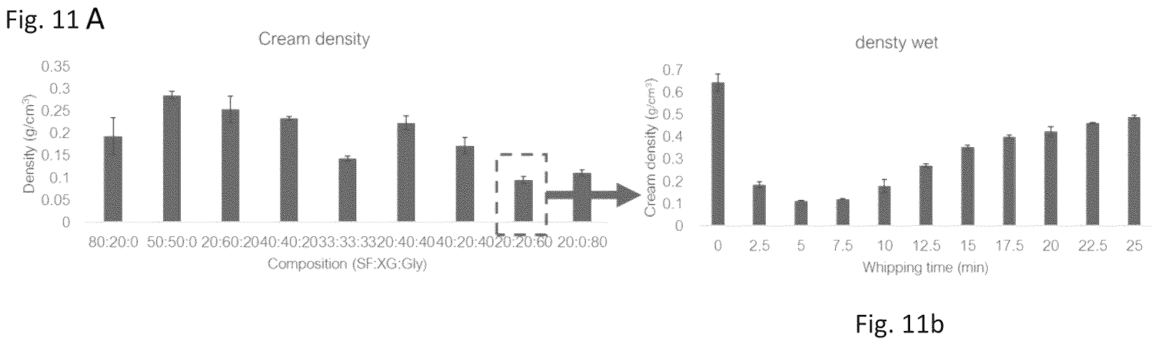

Example 6: Cream firmness (20:60:20): The mechanical properties of the cream (compression) were measured as a function of whipping time. FIGS. 11a and 11b depicts an increase of firmness as the air content increases.

Example 7: Overrun and Baked Cream characterization: The composition 20:20:60 (SF:XG:Gly) exhibited the lowest density, so a detailed analysis of the whipping time was conducted. The properties were investigated by whipping at 200 rpm using a 6-wire whip over a duration ranging from 0 to 25 minutes. In FIG. 11A, the density variation of the cream is illustrated as a function of whipping time. The density fluctuation is a result of the varying amount of air incorporated into the foam during the whipping phase.

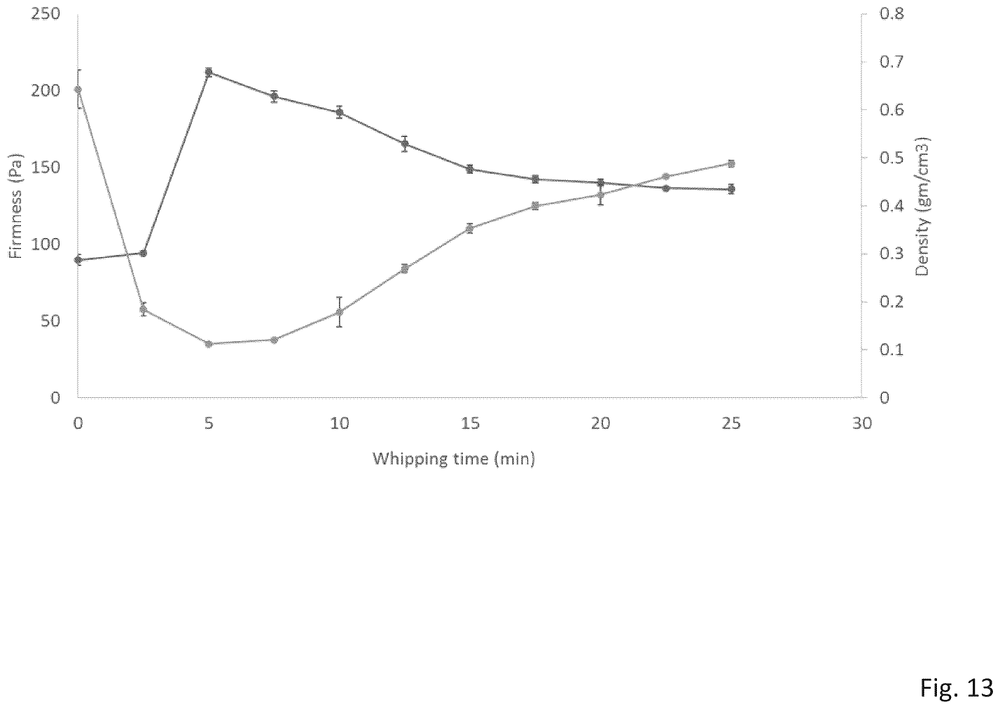

Additionally, the mechanical properties of the cream and their variation with whipping time through resistance toward compression (firmness) (FIG. 13) was measured. The findings indicate an increase in firmness as the density decreases or as the air content increases. The firmness of a cream, often indicative of its texture or consistency, holds significance not only in food applications but also in various industries. This quality is particularly relevant in sectors such as cosmetics and personal care products, pharmaceuticals, textile and fabric treatments, as well as art and craft supplies. With firmness controlled through whipping time, the silk cream exhibits a range of values spanning between 94 and 211 Pa, making it an appealing substrate for applications in cosmetics, pharmaceuticals, food, textiles, and fabrics. Given the mechanical properties of this silk foam, incorporating the silk cream into 3D printer materials was evaluated. The application of customized 3D food printing, for example, has gained attention in the culinary world for crafting intricate food designs and decorations. To indirectly gauge the air content of the cream, the overrun was evaluated, representing the increase in volume when air is incorporated during whipping. For the 20:20:60 composition, the volume increases up to 133% in 5 minutes (resulting in lower density) and then starts decreasing after 10 minutes due to excessive whipping. Similar to common whipped cream, over-whipping can lead to the separation of fat from the liquid, resulting in butter and buttermilk instead of whipped cream. This implies a parallel role played by silk in this process.

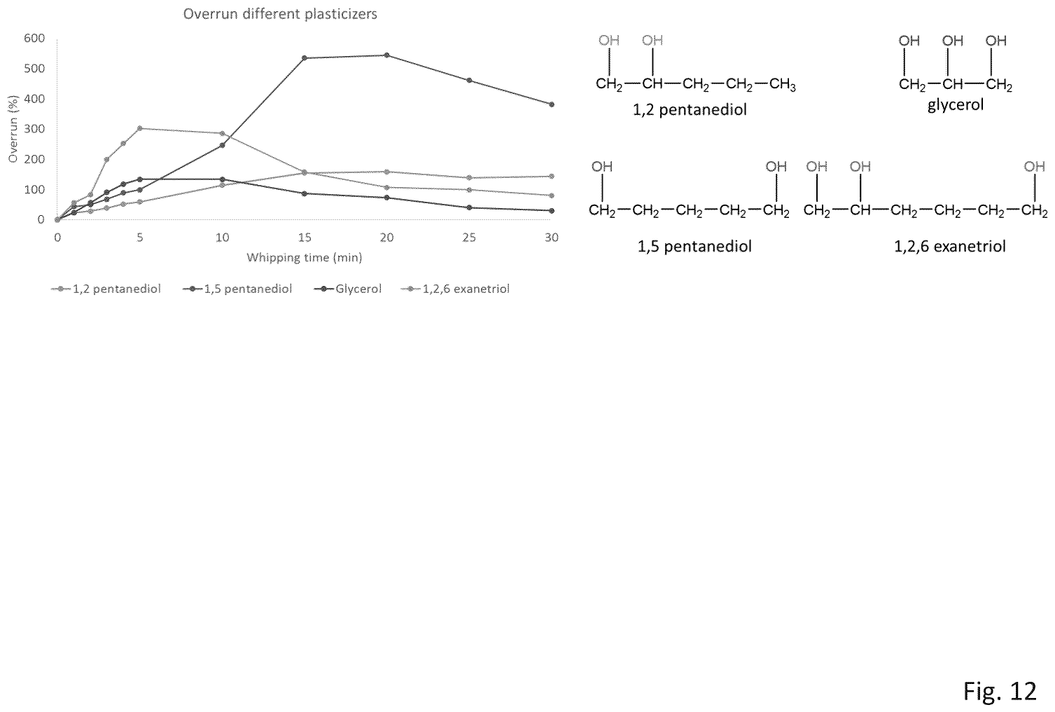

An additional method for controlling the cream's density/overrun involves using plasticizers other than glycerol. Overrun was measured using plasticizers with varying numbers or distances of —OH groups. The results show that the number of —OH groups affect the whipping time, while their distance influences the air capacity of the foam (FIG. 12). This phenomenon occurs because a hydrogen bonding network is formed between the protein and sugars during the whipping process, and plasticizers aid in bridging these interactions. As a result, a higher number of —OH functionalities (such as glycerol and 1-3-6 hexanetriol) lead to faster interactions, reaching the overrun plateau in just a few minutes. On the other hand, diols (1,2 and 1,5 pentanediol) exhibit slower cream growth (15-20 minutes) with a significantly higher overrun when the —OH groups are at a greater distance (150% vs. 500%).

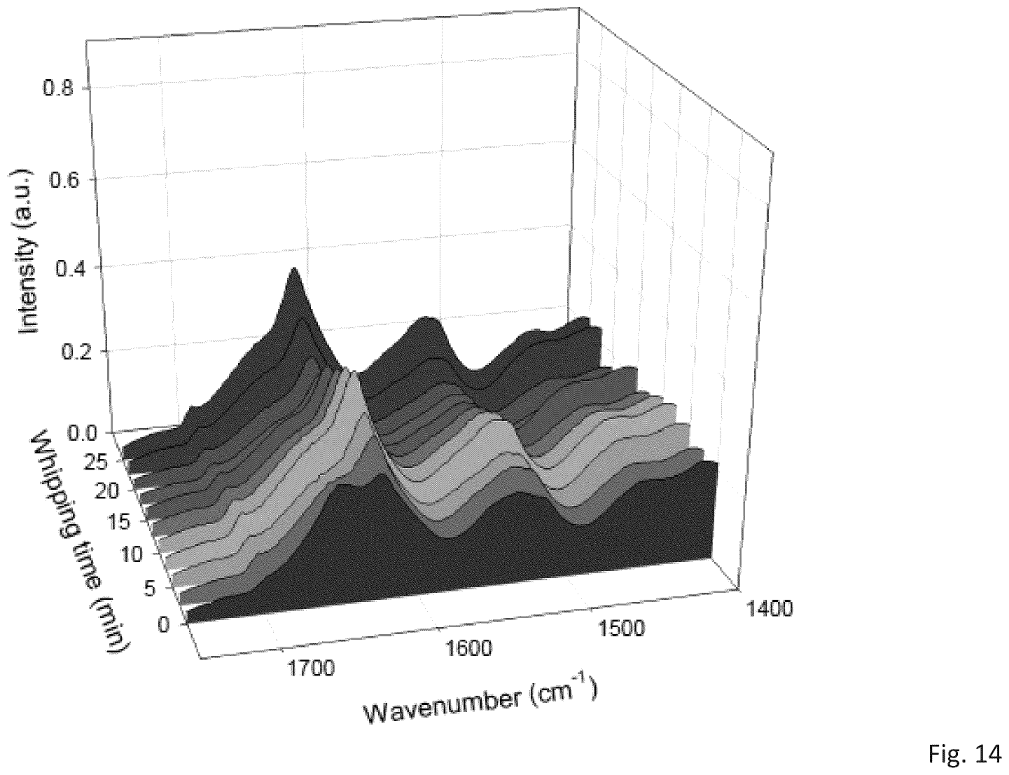

Example 8: Foams FTIR characterization: Unwhipped foams display a considerable amount of random coils, but after few minutes of whipping (e.g., 2.5 min), silk turns to β-sheet structure with no further variation for longer whipping times, as seen in FIG. 14. This is attributable to silk's shear thinning behavior.

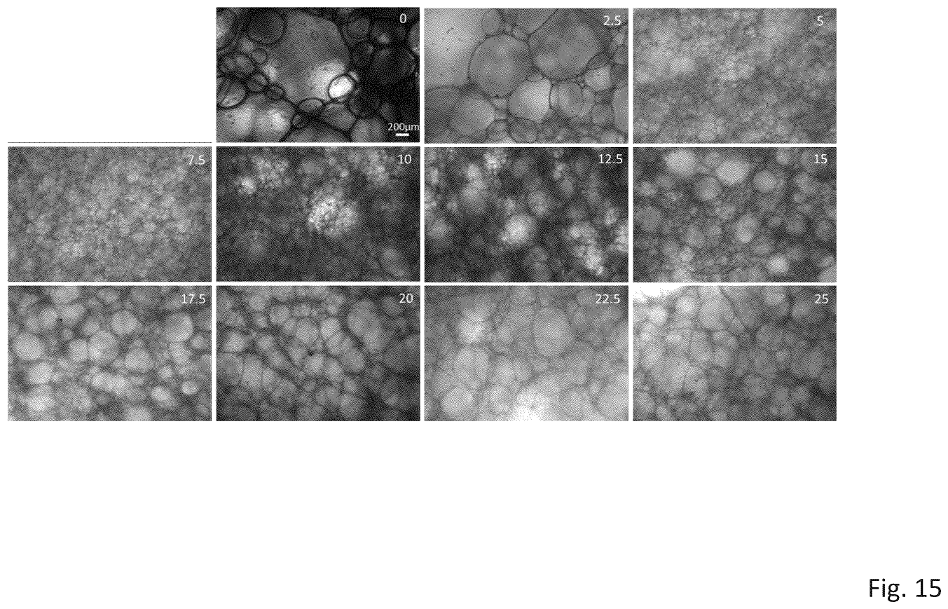

Example 9: Foams optical characterization: The average bubble size decreases in the initial phase (5-10 min) corresponding to the highest overrun value, and then increases again and remains relatively stable during prolonged whipping (20-25 min), as depicted in FIG. 15.

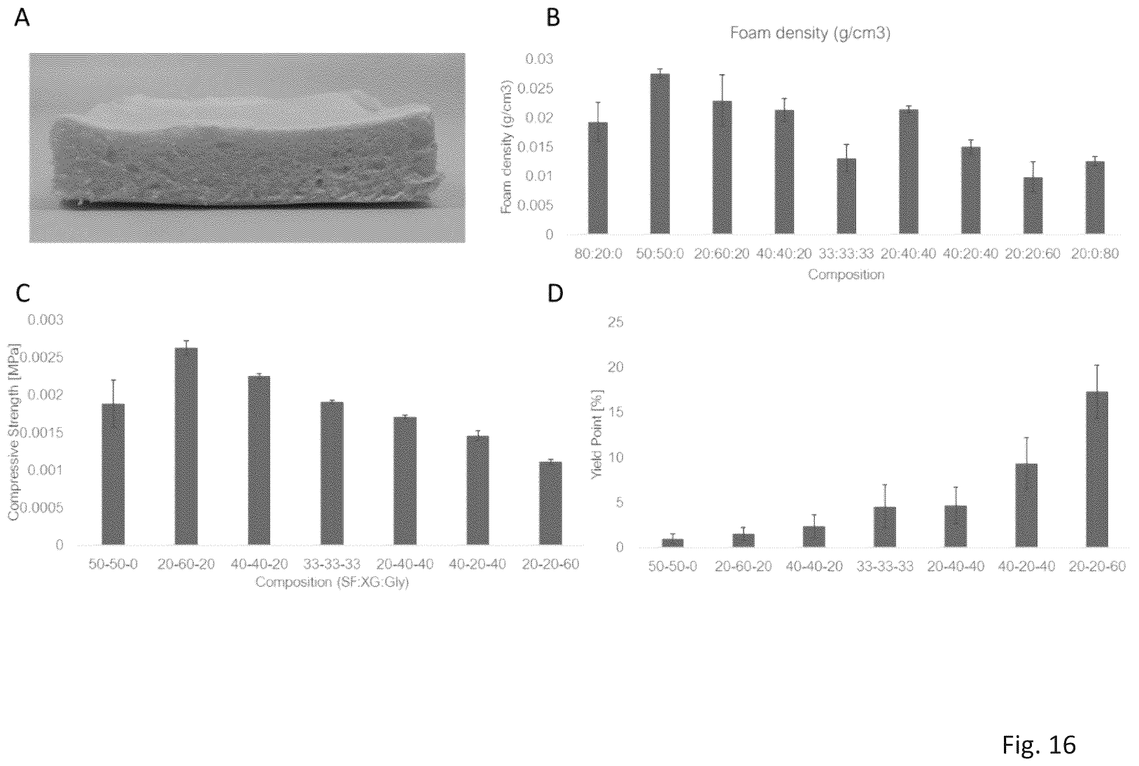

Example 10: Foam physical characterization: Composition related analyses: Tests were conducted on the physical and mechanical properties of the foams (baked creams, FIG. 16A) obtained with different composition ratios. The density decreased by a factor of 10 compared to its wet state (FIG. 16B). Regarding the mechanical properties, the foam's compressive strength and yield point (FIG. 16C, D) were assessed and found to be primarily influenced by the glycerol content.

In general, foams obtained with SF, XG, and glycerol are very soft and deformable, especially the 20:20:60 composition, making it ideal for a compressed material (the silk-leather). However, it may be less suitable for using the foam in its expanded state, as silk and xanthan gum foam tend to self-collapse within a few days.

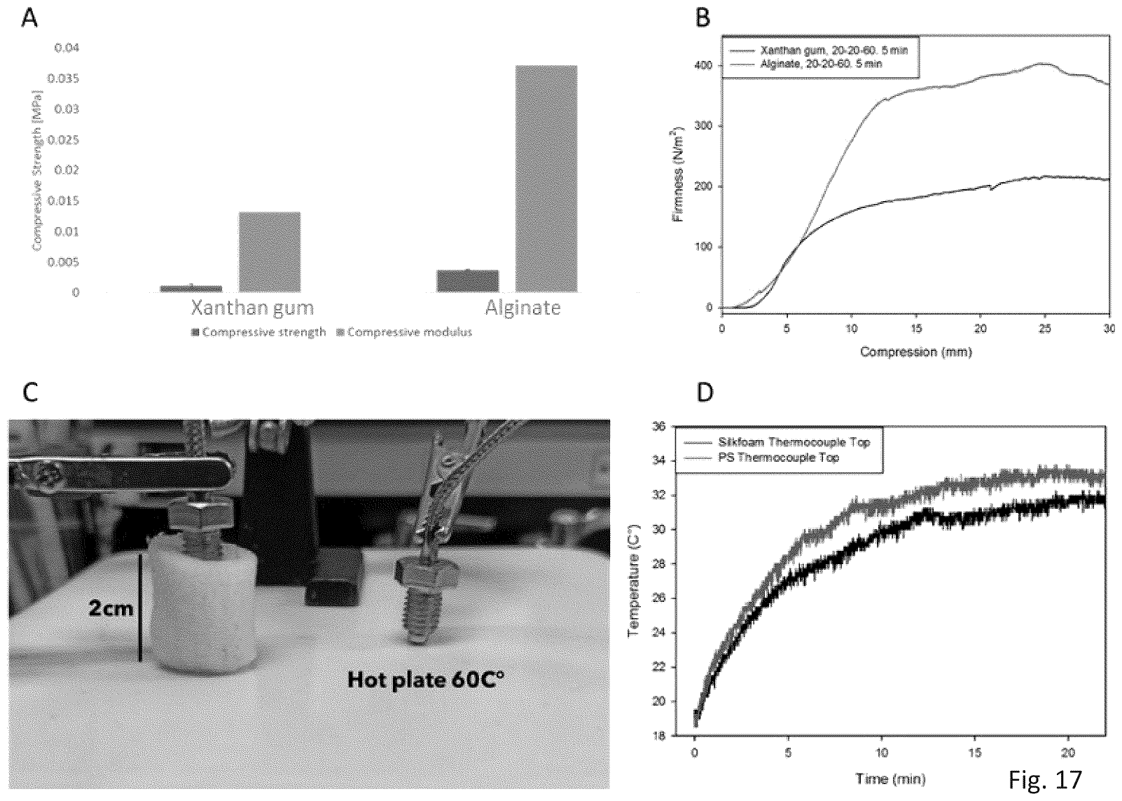

To enhance the mechanical properties of the dried foam for potential applications in their expanded state, alternative sugars may be considered. For instance, using alginate instead of xanthan gum significantly increases the mechanical performance of both dried foams (FIG. 17A) and creams (FIG. 17B). There may be additional applications of alginate-based foams in their expanded state, such as thermal insulation. In FIG. 17C, a setup for measuring the thermal conductivity of foam materials is presented. Briefly, two thermocouples are placed-one in contact with a hot plate with a surface temperature of 60° C. and the other at a 2 cm height with either the 20:20:60 silk-alginate foam or polystyrene in between—and the temperature is recorded in a time-resolved experiment. As shown in FIG. 17D, the silk-alginate foam displays a very similar trend to polystyrene, which is known to have a thermal conductivity between 0.034 and 0.038 W/mK. Additives such as borate ions may further increase mechanical properties while providing flame retardant and anti-fungal properties.

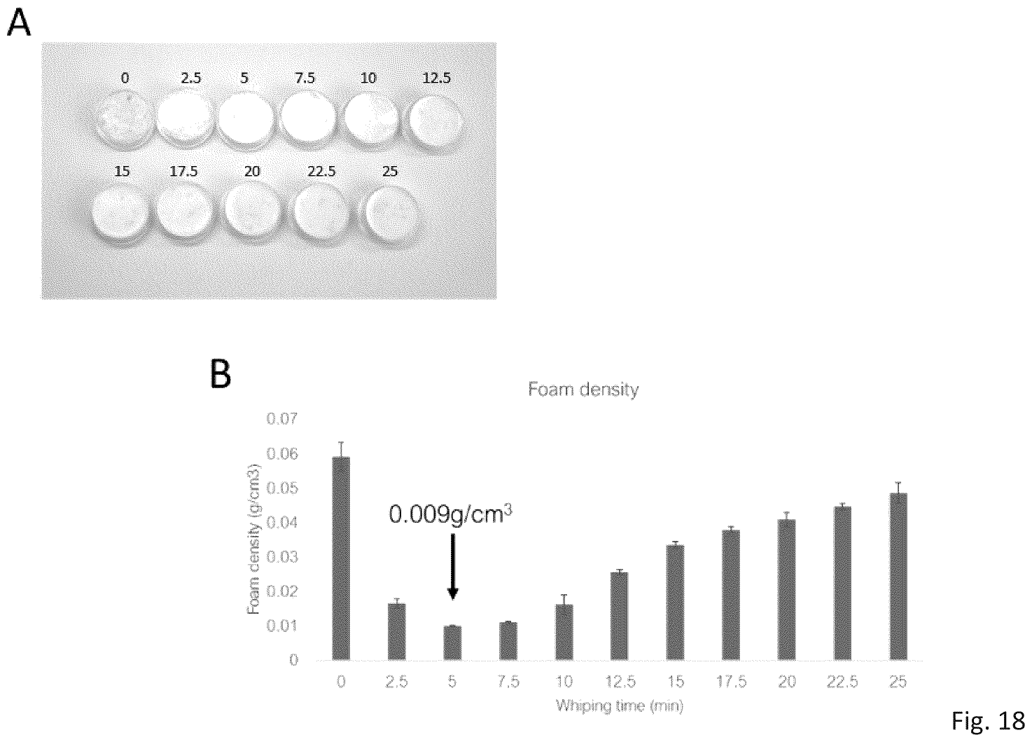

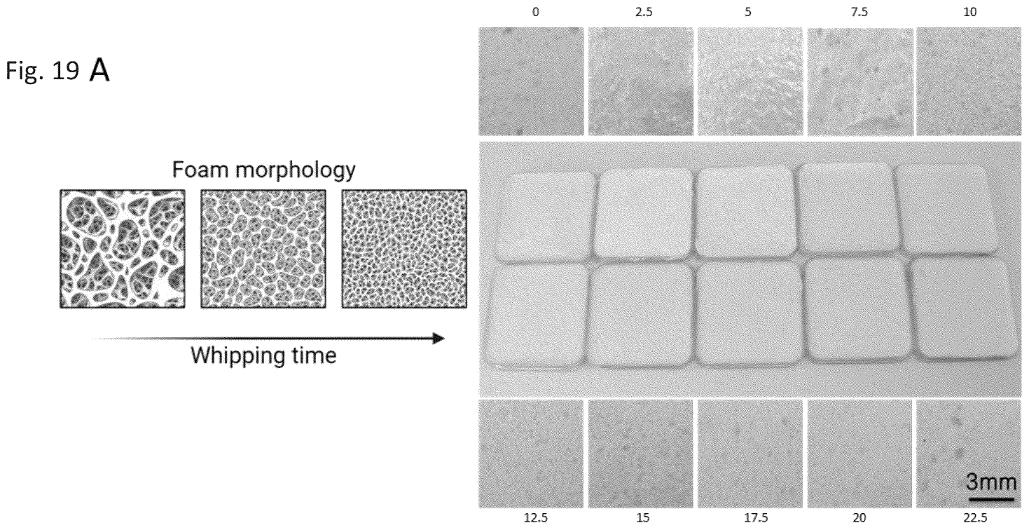

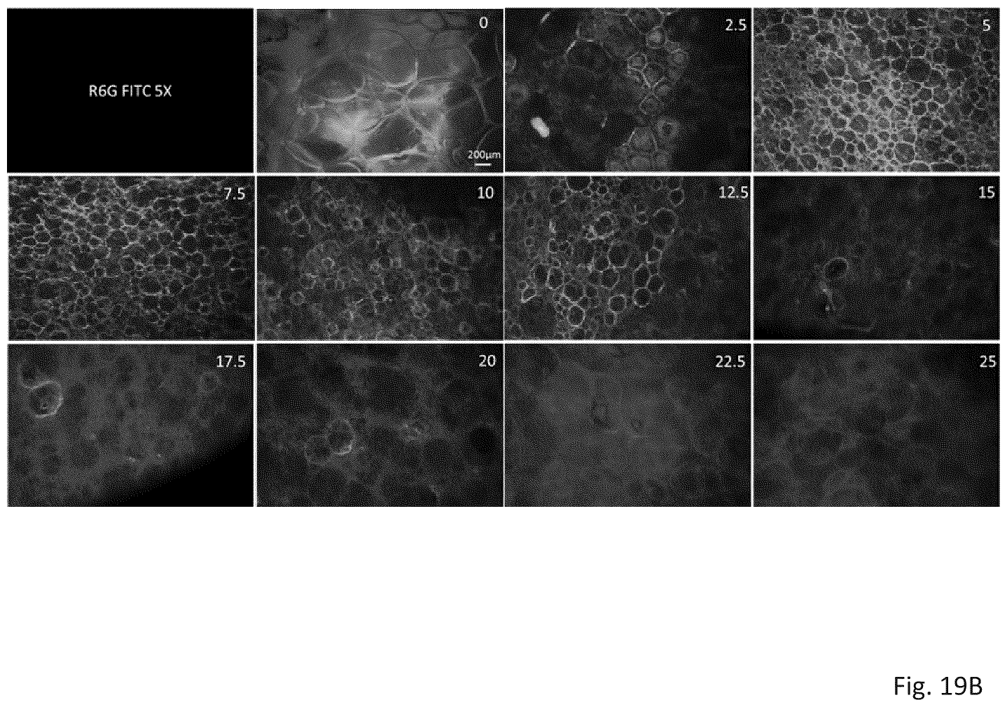

Example 11: Whipping time related analysis: The creams with various compositions, including the 20:20:60 composition obtained at different whipping times, were subsequently baked at 60° C. overnight and further characterized in their dried state. As depicted in FIG. 18A, the dried foams exhibit varying whiteness depending on the whipping time. This difference is likely attributed to the distribution of bubble sizes or the open/closed cell morphology, which may vary based on the whipping time (see FIG. 19). The density, once again, is one-tenth of the density in the wet state and can be adjusted by the whipping time, ranging from 0.009 to 0.048 g/cm³ (FIG. 18B). This value is notably low compared to other protein-based foams.

In FIG. 19A, schematics and pictures illustrating the surface detail are presented, showing the proposed change in the internal architecture of the foam at different whipping times. As previously mentioned, the whiteness and the reflective and scattering properties of the foams change with whipping time. This aspect may be significant for leather applications as it may influence the tactile sensation of the compressed material.

In FIG. 19B, the foam's internal structure was analyzed using fluorescent staining. It can be observed that the bubble size is polydisperse, but the average diameter ranges between millimeters and micrometers depending on the whipping duration, with variations in the internal structure corresponding to the overrun trend (the overrun value remains stable between 5 and 10 minutes, and so does the foam's internal structure). The smallest bubbles (around 100 μm) are obtained with a 7.5-minute whipping, and the cell structure transitions from a closed cell structure at 2.5 minutes to an open cell structure after 5 minutes of whipping.

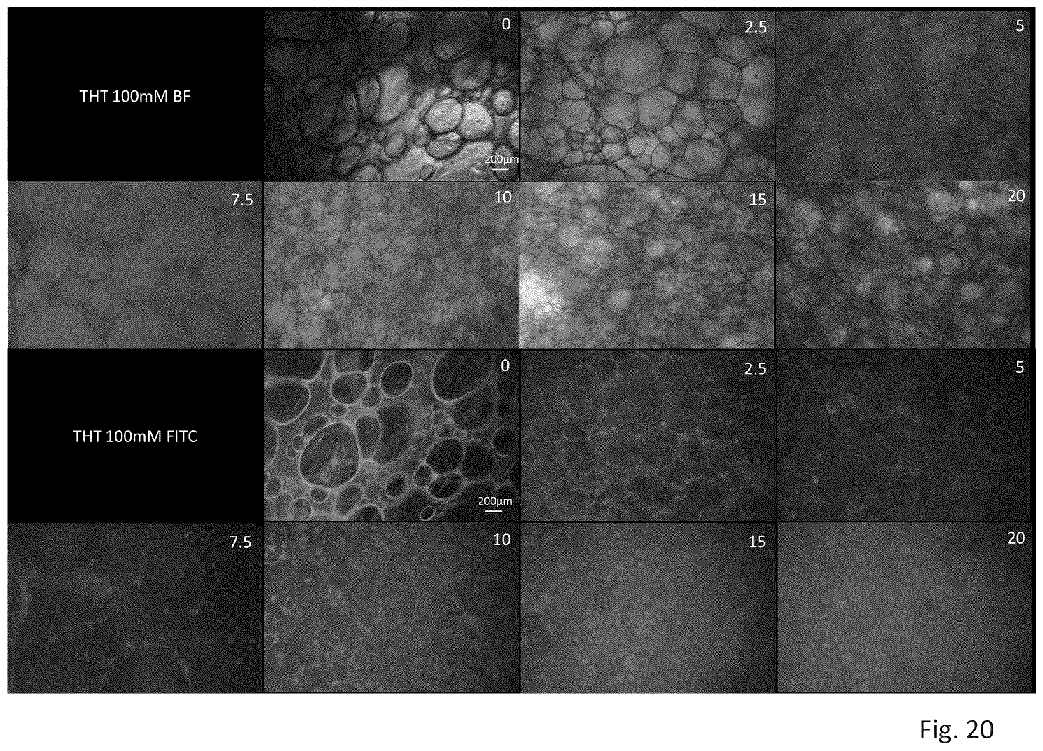

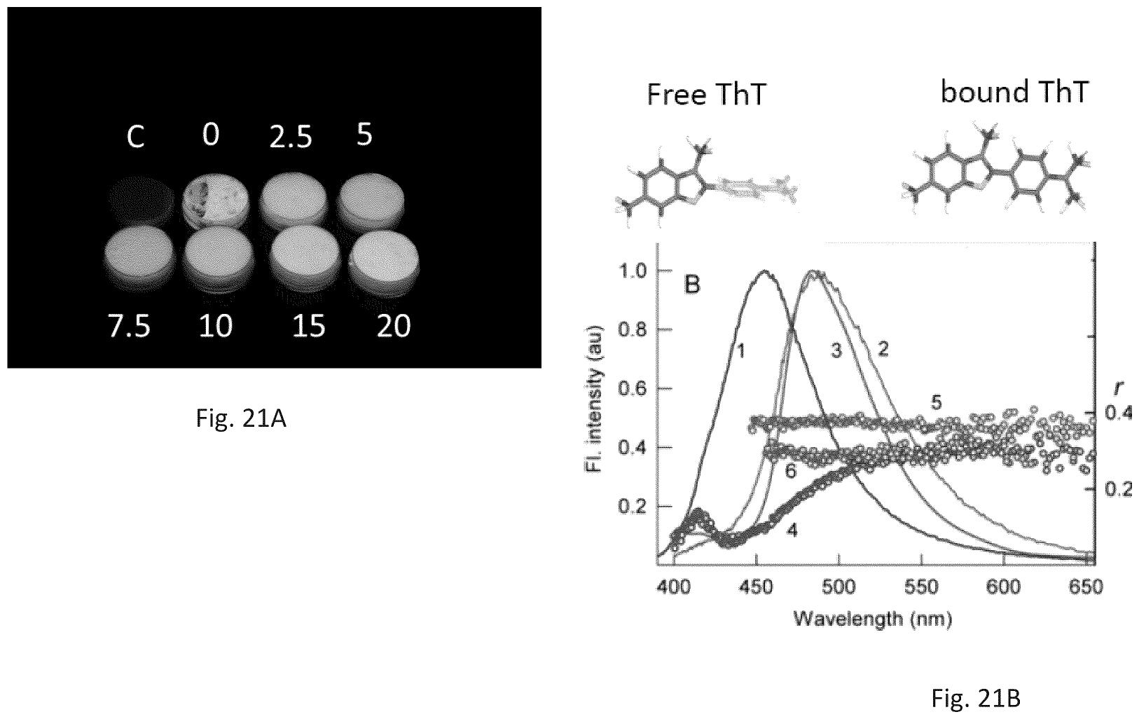

Example 12: Foams fluorescent staining (ThT): To better understand the internal foam structure, a fluorescent dye (ThT) was added (100 mM) during the whipping phase and the dried foams were analyzed in reflection with BF and FITC filter (FIG. 20). ThT was chosen because is a staining agent for β-amyloids and shows different spectral features if it is free in solution or bound to a β-sheet structure when excited at 365 nm (FIGS. 21A and 21B).

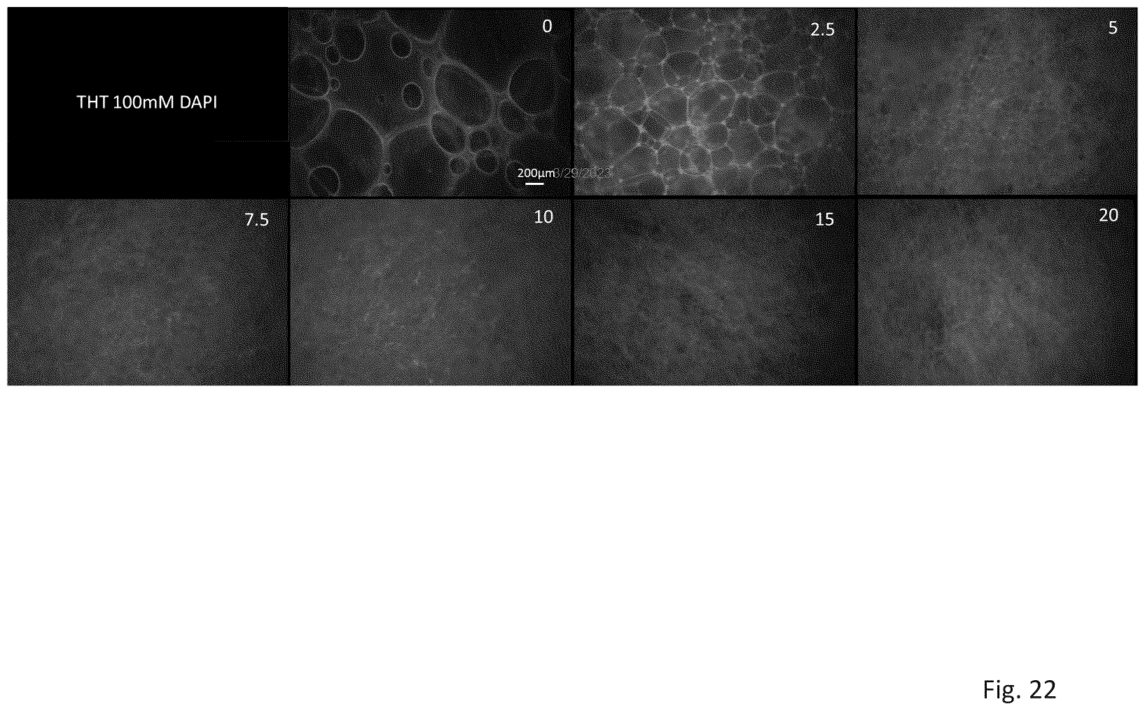

The emission of foams excited at 365 nm display a color shift during the initial whipping phases (FIG. 22). This is probably due to a different binding of the dye to the increasing β-sheet content in silk (due to its shear thinning behavior) and this is in accordance with the FTIR of baked foams.

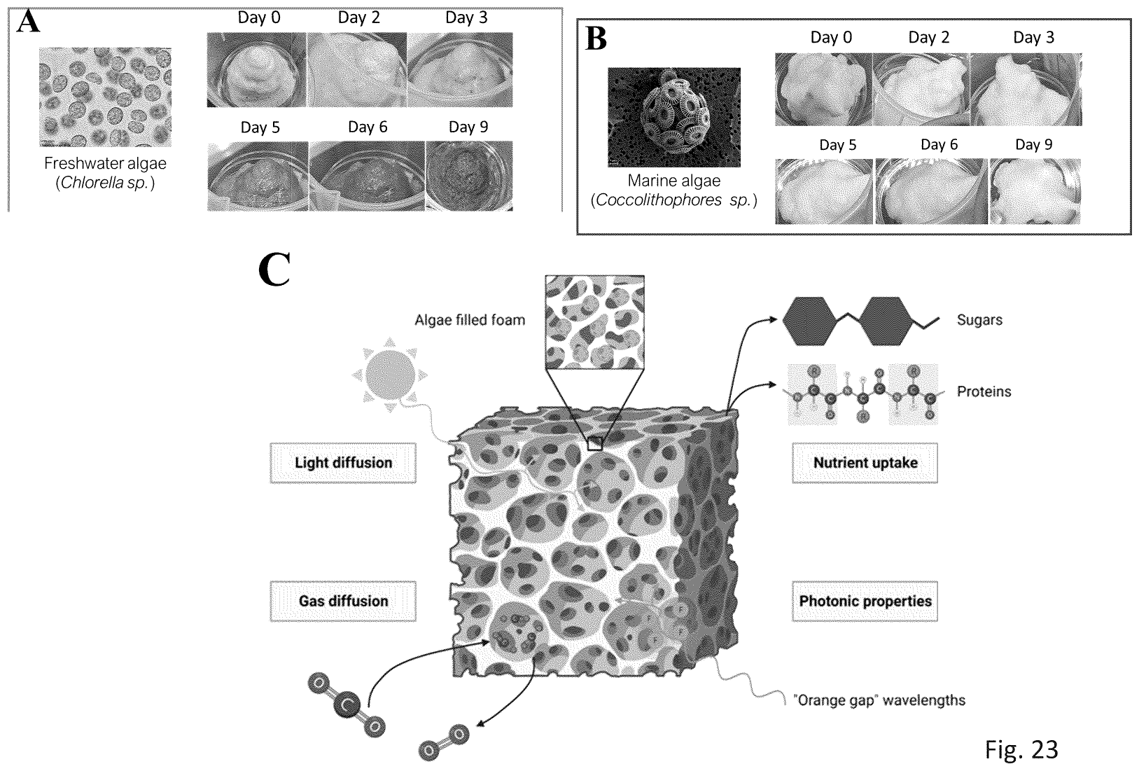

Example 13: Algal growth: Foam materials in their wet state (cream) may be used as a substrate for algal growth as shown in FIG. 23. Two algal strains from fresh water (chlorella) (FIG. 23A) and marine (coccolithophores) (FIG. 23B) environments were used to inoculate the cream. The creams were stored in a greenhouse at 90% of relative humidity and sunlight illumination for 9 days visibly observing cellular growth for both cases. Global photosynthesis consumes ten times more CO₂ than net anthropogenic emissions, and microalgae account for nearly half of this consumption. The high efficiency of algal photosynthesis relies on a mechanism concentrating CO₂, which enhances its fixation at a rate of 10-50 times higher than terrestrial plants. This high rate allows algae to convert up to 2.7 tons of CO₂ per acre per day, making them a good candidate for use in agriculture as a carbon-negative process that can also produce products. However, despite the potential benefits, the annual production of commercialized microalgal biomass (13,600 t) is low compared to worldwide CO₂ emissions (˜40 Gt year⁻¹). Therefore, new technologies need to be developed to increase productivity and make it economically viable. It has been reported that carbon dioxide fixation by microalgae might become economically viable when production costs fall below $500 t⁻¹ ha⁻¹ year⁻¹. A key factor in achieving this is improving the photosynthetic conversion efficiency, which is currently between 1.5% and 6%. Increasing this efficiency would allow for higher productivity and lower costs.

Microalgae have a wide range of diversity, and developing a substance to enhance their growth could help with the development of new technologies for different purposes such as direct carbon capture, hydrogen production, biofuels, food for humans or livestock, and biodegradation/bioremediation, each requiring a specific algal strain. For instance, coccolithophores are an interesting option for carbon capture applications. Coccolithophores are single-celled algae that have calcareous plates called coccoliths. These plates are formed through a biomineralization process where CO₂ is trapped as calcium carbonate, providing a permanent sink for carbon emissions. This makes them important in the marine carbon cycle and helps to mitigate the effects of greenhouse gas emissions. Other algae species fix CO₂ into organic polymers that are converted back into CO₂ in a short period, but coccolithophores provide a long-term solution for carbon storage. In order to improve the algal economy and overcome current technological limitations, a foam substrate for algal culturing may be used. This substrate has several advantages that can enhance algal growth (FIG. 23C). The structural material of the foam can be consumed by the algae, providing them with nutrients and other biomolecules. The porous structure of the foam can also help to distribute light and gas more uniformly, which are currently limiting factors in algal culturing techniques. Additionally, the photosynthesis process can be enhanced by adding artificial antennas (fluorophores) to the foam structure, which fill the chlorophyll's orange gap and increase the available solar spectrum. This approach could also allow for out-of-water cultivation, as it only requires a humid environment or a greenhouse, potentially expanding the possibilities for a broader bio-based economy.

1.-335. (canceled)

336. A compressed or hot pressed silk meringue comprising silk fibroin, a polysaccharide, and a plasticizer.

337. (canceled)

338. A method of making a composition, the method comprising whipping a liquid composition comprising silk fibroin, a polysaccharide, and a plasticizer for a predetermined whipping time to form a whipped silk cream;
baking the whipped silk cream at a temperature of between 30° C. and 150° C. for a length of time of between 5 minutes and 24 hours to form a silk meringue; and
compressing the silk meringue with a force of between 0.25 MPa and 25 MPa for a length of time of between 5 minutes and 6 hours to form a compressed silk meringue.

339-340. (canceled)

341. The method of claim 338, wherein the compressing is performed at an elevated temperature of between 80° C. and 200° C. and the compressed silk meringue is a hot-pressed silk meringue.

342. The method of claim 338, wherein the predetermined whipping time is between 5 minutes and 30 minutes.

343. (canceled)

344. The method of claim 338, wherein the baking is performed at a temperature of between 50° C. and 80° C. for a length of time of between 2 hours and 24 hours.

345. (canceled)

346. The method of claim 338, wherein the silk fibroin and polysaccharide are present in a weight ratio of between 1:4 and 20:1.

347. The method of claim 338, wherein the plasticizer is present in an amount by weight of between 0.5% and 75.0%.

348. The method of claim 338, the liquid composition further comprising at least one of a sensing agent, a therapeutically active agent, a colorant, or an aromant.

349. The method of claim 338, wherein the polysaccharide is an alginate or xanthan gum.

350. The method of claim 338, wherein the plasticizer is selected from the group consisting of glycerol, 1,2 pentanediol, 1,5 pentanediol, 1,2,6 hexanetriol, and combinations thereof.

351-369. (canceled)

370. A method of making a whipped silk cream having a desired whipped silk cream morphology, a silk meringue having a desired silk meringue morphology, a compressed silk meringue having a desired compressed silk meringue morphology, or a hot-pressed silk meringue having a desired hot-pressed silk meringue morphology, the method comprising:
selecting a silk concentration, a silk molecular weight distribution, a polysaccharide species, a polysaccharide concentration, a plasticizer species, a plasticizer concentration, a whipping speed, optionally a silk meringue baking temperature, optionally a compressed silk meringue compressing force, optionally a hot-pressed silk meringue hot-pressing force and temperature; and
making the whipped silk cream, the silk meringue, the compressed silk meringue, or the hot-pressed silk meringue using the silk concentration, the silk molecular weight distribution, the polysaccharide species, the polysaccharide concentration, the plasticizer species, the plasticizer concentration, the whipping speed, optionally the silk meringue baking temperature, optionally the compressed silk meringue compressing force, and optionally the hot-pressed silk meringue hot-pressing force and temperature.

371. The method of claim 338, the method further comprising: adhering the compressed silk meringue to a fabric, thereby forming a silk leather.

372. The method of claim 338, wherein the polysaccharide is xanthan gum.

373. The method of claim 338, wherein the polysaccharide is an alginate.

374. The method of claim 338, wherein the plasticizer is glycerol.

375. The method of claim 341, wherein the hot-pressed silk meringue has a water content of between 2% and 50%.

376. The method of claim 338, the method further comprising embossing a surface of the compressed silk meringue.

377. A compressed silk meringue made by the method of claim 338.

378. A hot-pressed silk meringue made by the method of claim 341.

379. A silk leather made by the method of claim 371.
